(19) United States
(12) Patent Application Publication
(10) Pub. No.: US 2020/0197281 A1
(43) Pub. Date: Jun. 25, 2020

(54) OXIDATION COLORING AGENT IN RED SHADES WITH REDUCED COLOR SHIFT DUE TO COPPER IONS

(71) Applicant: Henkel AG & Co. KGaA, Duesseldorf (DE)

(72) Inventors: JING HODES, Hagen (DE); DANIELA KESSLER-BECKER, Leverkusen (DE)

(73) Assignee: Henkel AG & Co. KGaA, Duesseldorf (DE)

(21) Appl. No.: 16/693,931

(22) Filed: Nov. 25, 2019

(30) Foreign Application Priority Data
Dec. 19, 2018 (DE) 10 2018 222 222.3

(51) Int. Cl. A61K 8/49; A61K 8/44; A61K 8/22; A61Q 5/10

(52) U.S. Cl. CPC A61K 8/494; A61K 8/44; A61K 2800/884; A61Q 5/10; A61K 2800/4324; A61K 8/22

(57) ABSTRACT

An agent for the oxidative dyeing of keratinic fibers, in particular human hair, contains in a cosmetic carrier (A) at least one oxidation dye precursor with 4,5-diaminopyrazole as the basic structural element, (B) iminodisuccinic acid or a physiologically compatible salt, and (C) at least one oxidizing agent different from atmospheric oxygen.

### CROSS-REFERENCE TO RELATED APPLICATION

This application claims priority to German Patent Application No. 10 2018 222 222.3, filed Dec. 19, 2018, which is incorporated herein by reference in its entirety.

### TECHNICAL FIELD

The subject of the present application is an agent for the oxidative dyeing of keratinic fibers, in particular human hair.

### BACKGROUND

The change in the color of keratinic fibers, in particular hair, represents an important area of modern cosmetics. As a result, the appearance of the hair can be adapted to both current fashion trends and the individual wishes of the individual. The person skilled in the art knows different possibilities for changing the color of hair. Hair color can be changed temporarily through the use of direct acting dyes. In this case, already fully formed dyes diffuse from the coloring agent into the hair fiber. The dyeing with direct acting dyes is associated with little hair damage, but a disadvantage is the low durability and fast washability of the colorings obtained with direct acting dyes.

If the consumer wishes to have a long-lasting color result or a shade lighter than his original hair color, oxidative color-change agents are usually used. So-called oxidation coloring agents are used for permanent, intensive dyeings with corresponding fastness properties. Such coloring agents usually contain oxidation dye precursors, so-called developer components (oxidation bases) and coupler components which form the actual dyes with one another under the influence of oxidizing agents. Oxidation coloring agents are characterized by long-lasting coloring results.

Extensive prior art already exists for oxidative coloring agents. In particular, many attempts have been made to optimize the color intensity and the fastness properties of fashion shades.

However, despite the large number of optimization experiments already carried out, there is still need for improvement with regard to the fastness properties of the dyeing of oxidatively dyed keratin fibers, in particular when they are to be dyed in a fashion shade in the red region.

A problematic constituent that can negatively affect color development in oxidative hair dyeing is copper ions. For example, copper ions are present in tap water when copper pipes are used for its supply, or in swimming pools where copper sulfide is used as the algaecide. For example, the hair can come into contact with copper ions when bathing in swimming pools or washing hair with water flowing through copper pipes. Both the already dyed hair and the initial dyeing affect color development, especially a difference between the colors that are formed in the presence or absence of copper ions.

Besides the degradation of the melanin pigments, the oxidative treatment also attacks the polypeptide chains of the keratin fibers, in particular of the hair. Keratin fibers or hair that have already been oxidatively dyed or blonded several times are rougher, more brittle and more porous and thus more sensitive to environmental influences. Their absorption capacity is also increased. If they come into contact with copper ions, they are absorbed by the keratin fibers or hair. It forms a blue copper-protein complex that results with the natural or artificial hair color or as part of a renewed dyeing with the oxidation dye precursors mixed colors.

In naturally dark hair and in dyeing dark shades, copper ion absorption does not noticeably affect color color. In contrast, when dyeing in red shades, copper ion absorption can cause color shifts that are visible to the human eye.

Various products for treating unwanted copper ion absorption in keratin fibers, in particular hair, are known from the prior art. These include, for example, hydroxycarboxylic acids or complexing agents, such as hair cleaning products containing iminodisuccinic acid, as disclosed in WO2006/066641A1 and WO2008/043654A2. WO 2002/074272A1 teaches the use of complexing agents, such as iminodisuccinic acid, in higher concentrations in oxidative coloring and bleaching agents to reduce hair damage. EP 1462093A2 discloses oxidative coloring agents containing complexing agents, such as iminodisuccinic acid, for improving the homogeneity or reducing the selectivity of hair dyeing. The hair fiber is severely damaged from the hair root to the hair tip. Hair in the region of the neck has recently regrown and has been exposed to no or only little weather influences, chemical (dyeing, blonding, perming, washing, swimming pool water) or physical (combing, blow-drying) influences. The range of hair lengths is such that the farther away from the hairline and thus the older it is, the more damaged the hair. The hairs in the region of the tips are the oldest parts of the hair and therefore have the strongest damage. In damaged hair, the cuticle, the cuticle layer of the hair, is more or less destroyed. As a result, damaged hair generally has a stronger color lift. Therefore, when the base and tips are dyed with the same coloring agent, more damaged hair is always at risk of uneven color results. However, the problem of selectivity has nothing to do with the problem of color shift due to copper ions.

### BRIEF SUMMARY

In one embodiment, an agent for the oxidative dyeing of keratinic fibers comprises, in a cosmetic carrier (A), at least one oxidation dye precursor with 4,5-diaminopyrazole as the basic structural element according to the structural formula (I) and further (B) iminodisuccinic acid and/or at least one of its physiologically compatible salts. The agent as contemplated herein furthermore contains (C) at least one oxidizing agent which is different from atmospheric oxygen,

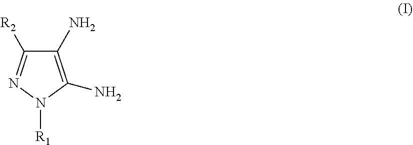

wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen

A second subject of the present disclosure is a multi-component packaging unit (kit-of-parts) comprising at least two separately prepared components, wherein the first component (K1) contains the above-described ingredients (A) and (B) and the second component (K2) contains the above-described ingredient (C).

A third subject of the present disclosure is a multi-component packaging unit (kit-of-parts) comprising at least two separately prepared components, wherein the first component (K1) contains the above-described ingredient (A) and optionally the above-described ingredient (C) and the second component (K2) contains the above-described ingredient (B).

A fourth subject of the present disclosure is a multi-component packaging unit (kit-of-parts) comprising at least three separately prepared components, wherein the first component (K1) contains the above-described ingredient (A), the second component (K2) contains the above-described ingredient (B) and the third component (K3) contains the above described ingredient (C).

A further subject of the present disclosure is a method for oxidative hair dyeing, in which an agent for oxidative dyeing of keratinic fibers, in particular human hair, is applied to the fibers, in particular human hair, which contains, in a cosmetic carrier (A), at least one oxidation dye precursor with 4,5-diaminopyrazole as the basic structural element according to the structural formula (I), furthermore (B) iminodisuccinic acid and/or at least one of its physiologically compatible salts and furthermore (C) at least one oxidizing agent other than atmospheric oxygen, the coloring agent being rinsed with water after an exposure time of from about 1 to about 60 minutes and the hair being optionally treated with further cleaning and care products and then dried.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was to provide oxidative coloring agents for achieving red shades based on 4,5-diaminopyrazoles as a developer component with reduced color shift due to copper ions.

In the context of the present application, the reduction of the color shift of a color shade due to copper ions is understood to mean the difference between the color which forms with a 4,5-diaminopyrazole as a developer component under the influence of hydrogen peroxide in the presence of copper ions and the color which forms with a 4,5-diaminopyrazole as a developer component under the influence of hydrogen peroxide in the absence of copper ions.

Keratinic fibers can be dyed in red shades when, in the coloring agents (A), at least one oxidation dye precursor of structure (I) and/or one of its physiologically compatible salts

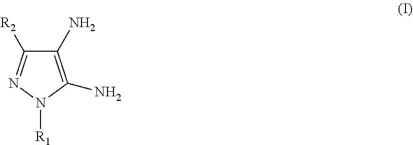

wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, is present as a developer component or oxidation base.

Preferred oxidation dye precursors of the structure (I) as contemplated herein are those in which R₁ stands for a linear or branched C1-C10 alkyl group which can be substituted by one to ten hydroxyl groups and R₂ stands for hydrogen.

Particularly preferred as contemplated herein is the oxidation dye precursor of the structure (I) in which R₁ stands for a 2-hydroxyethyl group and R₂ stands for hydrogen, that is, 4,5-diamino-1-(2-hydroxyethyl)-1H-pyrazole and/or one of its physiologically compatible salts, preferably 4,5-diamino-1-(2-hydroxyethyl)-1H-pyrazole sulfate.

Also very preferred as contemplated herein is the oxidation dye precursor of the structure (I) in which R₁ stands for an n-hexyl group and R₂ stands for hydrogen. Oxidative coloring agents containing this dye 4,5-diamino-1-hexylpyrazole are known, for example, from WO2016177344A1.

Also very preferred as contemplated herein is the oxidation dye precursor of the structure (I) in which R₁ stands for an n-heptyl group and R₂ stands for hydrogen.

The developer component 4,5-diamino-1-(2-hydroxyethyl)-1H-pyrazole has long been used in many oxidation coloring agents. Corresponding agents are known, for example, from EP 1321131A2.

Surprisingly, it has been found that with the combination of (A), at least one oxidation dye precursor with 4,5-diaminopyrazole as the basic structural element according to the structural formula (I), and furthermore (B), iminodisuccinic acid and/or at least one of its physiologically compatible salts in a cosmetic carrier, oxidation coloring agents are obtained which provide attractive and fashionable shades of red with precise predictability of the color result regardless of the copper ion content of the hair.

Oxidation coloring agents containing iminodisuccinic acid or tetrasodium iminodisuccinate (INCI: Tetrasodium iminodisuccinate) are known in the art from WO 2002/074272A1 and EP 1462093A2. None of these documents discloses a content of 4,5-diaminopyrazoles or the use of iminodisuccinic acid or tetrasodium iminodisuccinate for reducing the color-shifting effect of copper ions.

A first subject of the present disclosure is therefore an agent for the oxidative dyeing of keratinic fibers, in particular human hair, containing in a cosmetic carrier

(A) at least one oxidation dye precursor of the structure (I) and/or one of its physiologically compatible salts

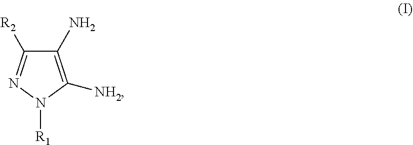

wherein R₁ and R₂, independently of one another, stand for hydrogen or a linear or branched C₁-C10 alkyl group which can be substituted by one to ten hydroxyl groups, wherein R₁ and R₂ do not simultaneously stand for hydrogen, further
(B) iminodisuccinic acid and/or at least one of its physiologically compatible salts, and
(C) at least one oxidizing agent other than atmospheric oxygen.

Keratinic fibers, keratin-containing fibers or keratin fibers are understood to mean furs, wool, feathers and, in particular, human hair. Although the agents as contemplated herein are primarily suitable for dyeing keratin fibers, in principle, there is nothing to prevent their use in other fields as well.

The term “agent for oxidative dyeing” as used in the present disclosure is understood to mean oxidative coloring agents containing development type and coupler type of oxidation dye precursor. The formation of the dyeing is carried out by the presence of an oxidizing agent (C) which is different from atmospheric oxygen and which is preferably hydrogen peroxide. Depending on the amount of oxidizing agent used, the keratin fiber is at the same time lightened more or less strongly during dyeing, since the oxidizing agent not only initiates the dye-forming process of developers and couplers but also oxidatively destroys the hairs' own pigments (melanins).

The agents as contemplated herein contain the at least one oxidation dye precursor (A) of the structure (I) and also component (B) and the oxidizing agent (C) in a cosmetic carrier, preferably in a suitable aqueous, alcoholic or aqueous-alcoholic carrier. For the purpose of oxidative dyeing, such carriers can be, for example, creams, emulsions, gels or even foaming solutions such as, for example, shampoos, foam aerosols, foam formulations or other preparations which are suitable for use on the hair. Particularly preferred agents for the oxidative dyeing of keratinic fibers are creams or emulsions.

Characteristic of the agents as contemplated herein is a content of at least one oxidation dye precursor (A) of the structure (I) and/or one of its physiologically compatible salts

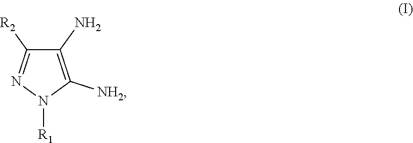

wherein R₁ and R₂, independently of one another, stand for hydrogen or a linear or branched C₁-C₁₀ alkyl group which can be substituted by one to ten hydroxyl groups, wherein R₁ and R₂ do not simultaneously stand for hydrogen.

In the context of the present disclosure, a developer is understood to mean a developer-type oxidation dye precursor. In the context of the present disclosure, a coupler is understood to mean a coupler-type oxidation dye precursor.

Extraordinarily preferred agents as contemplated herein contain 4,5-diamino-1-(2-hydroxyethyl)-1H-pyrazole and/or one of its physiologically compatible salts as the oxidation dye precursor (A).

4,5-diamino-1-(2-hydroxyethyl)-1H-pyrazole is the compound of the formula (I-A), that is, an oxidation dye precursor of the structure (I) in which R₁ stands for a 2-hydroxyethyl group and R₂ stands for hydrogen, that is, 4,5-diamino-1-(2-hydroxyethyl)-1H-pyrazole and/or one of its physiologically compatible salts:

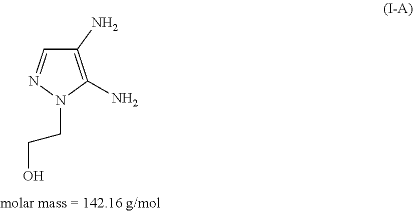

Preferred physiologically compatible salts of 4,5-diamino-1-(2-hydroxyethyl)-1H-pyrazole are in particular the hydrochlorides (monohydrochloride×HCl, or dihydrochloride×2 HCl), the sulfate (x H₂SO₄) and the hydrobromides (monohydrobromide×HBr, or dihydrobromide×2 HBr) of the compound. Very particular preference is given to 4,5-diamino-1-(2-hydroxyethyl)-1H-pyrazole sulfate (Formula (III)).

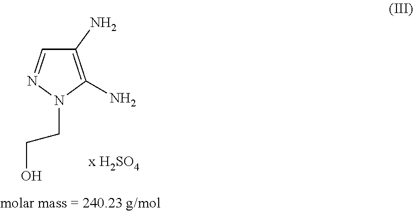

Further preferred agents as contemplated herein contain, as an oxidation dye precursor (A), at least one oxidation dye precursor of the structure (I) in which R₁ stands for an n-hexyl group and R₂ stands for hydrogen (4,5-diamino-1 hexyl-1H-pyrazol).

Further preferred agents as contemplated herein contain, as an oxidation dye precursor (A), at least one oxidation dye precursor of the structure (I) in which R₁ stands for an n-heptyl group and R₂ stands for hydrogen (4,5-diamino-1-heptyl-1H-pyrazol).

Agents preferred for oxidative dyeing are characterized in that the at least one oxidation dye precursor (A) of the structure (I) is present in a total amount of from about 0.01 to about 2.5% by weight, preferably from about 0.1 to about 1.8% by weight, more preferably from about 0.2 to about 1.0% by weight, most preferably from about 0.4 to about 0.9% by weight, wherein the amount specifications are based on the weight of the free 4,5-diaminopyrazole base in relation to the weight of the agent as contemplated herein.

Particularly preferred agents for oxidative dyeing are characterized in that 4,5-diamino-1-(2-hydroxyethyl)-1H-pyrazole (structure I-A) is present in a total amount of from about 0.01 to about 2.5% by weight, preferably from about 0.1 to about 1.8% by weight, particularly preferably from about 0.2 to about 1.0% by weight, most preferably from about 0.4 to about 0.9% by weight, wherein the amount specifications by weight refer to the free 4,5-diaminopyrazole base in relation to the weight of the agent as contemplated herein. 4,5-diamino-1-(2-hydroxyethyl)-1H-pyrazole (I-A) is most preferably used in the form of the sulfate salt in an amount of from about 0.025 to about 4.5% by weight, preferably from about 0.1 to about 3.5% by weight, more preferably from about 0.2 to about 2.0% by weight and very particularly preferably from about 0.3 to about 1.0% by weight. In this case, the amount is based on the weight of 4,5-diamino-1-(2-hydroxyethyl)-1H-pyrazole sulfate (Formula (III)), which is adjusted in relation to the weight of the agent as contemplated herein.

In a particularly preferred embodiment, an agent as contemplated herein is therefore characterized in that, based on its total weight, it contains (A) from about 0.025 to about 4.5% by weight, preferably from about 0.1 to about 3.5% by weight, more preferably from about 0.2 to about 2.0% by weight and most preferably from about 0.3 to about 1.0% by weight of 4,5-diamino-1-(2-hydroxyethyl)-1H-pyrazole sulfate.

Further preferred agents for oxidative dyeing oxidative as contemplated herein are characterized in that 4,5-diamino-1-hexyl-1H-pyrazole is present from about 0.01 to about 2.5% by weight, preferably from about 0.1 to about 1.8% by weight, particularly preferably from about 0.2 to about 1.0% by weight, most preferably from about 0.4 to about 0.9% by weight, wherein the amounts are based on the weight of the free 4,5-diaminopyrazole base in relation to the weight of the agent.

The agents as contemplated herein contain iminodisuccinic acid and/or at least one of its physiologically tolerated salts as a second essential constituent (B).

Iminodisuccinic acid (about 249.1 g/mol) is also denoted as N-(1,2-dicarboxyethyl) aspartic acid. In the context of the present disclosure, the physiologically compatible salts of iminodisuccinic acid include in particular the salts of alkali metals, in particular of lithium, sodium and potassium, more preferably sodium and potassium, most preferably sodium. The extremely preferred as contemplated herein tetrasodium iminodisuccinate (INCI: Tetrasodium iminodisuccinate; about 337.10 g/mol) is commercially available.

Agents for oxidative dyeing preferred as contemplated herein are characterized in that iminodisuccinic acid and/or at least one of its physiologically compatible salts is/are present in a total amount of from about 0.01 to about 3% by weight, preferably from about 0.05 to about 2% by weight, more preferably from about 0.1 to about 1.5% by weight, exceptionally preferably from about 0.3 to about 1.0% by weight, wherein the amounts refer to the weight converted to free iminodisuccinic acid in relation to the weight of the agent as contemplated herein.

The agent as contemplated herein for oxidative dyeing of keratinic fibers (C) contains at least one oxidizing agent other than atmospheric oxygen as a third essential constituent.

Oxidizing agents (C) preferred as contemplated herein are selected from hydrogen peroxide, sodium percarbonate, percarbonates and persalts, in particular peroxodisulfate salts and/or peroxomonosulfate salts, and mixtures thereof, wherein hydrogen peroxide is particularly preferred as contemplated herein.

As soon as the at least one oxidation dye precursor (A) and optionally further optional oxidation dye precursors come into contact with the oxidizing agent (C) in the presence of water, a coupling process starts and the dye formation begins.

The agent as contemplated herein, which contains the ingredients (A), (B) and (C), is therefore the ready-to-use oxidative coloring agent, if the oxidizing agent (C) comprises hydrogen peroxide.

Agents as contemplated herein containing ingredients (A), (B) and (C), wherein the oxidizing agent (C) alone is selected from sodium percarbonate, percarbonates and persalts, in particular peroxodisulfate salts and/or peroxomonosulfate salts, require a water content in order to activate the oxidizing agent (C) to start the coupling process of the oxidation dye precursors and the associated dye formation.

The amount of oxidizing agent is chosen by one skilled in the art depending on the desired lightening performance. When the formation of a very dark red shade is desired, the person skilled in the art will reduce the amount of hydrogen peroxide to be used accordingly. However, if a bright red shade on dark hair is to be achieved, the hair must also be lightened to a significant extent at the same time. In this case, the amount of hydrogen peroxide used is chosen to be high; optionally, a persalt, in particular one or more peroxodisulfate salts and/or peroxomonosulfate salts, such as potassium persulfate, sodium persulfate or ammonium persulfate, can be present as a further oxidizing agent (C) for this case.

Agents preferred as contemplated herein, based in each case on their weight, contain from about 0.5 to about 12% by weight, preferably from about 0.9 to about 7% by weight, particularly preferably from about 1.5 to about 5% by weight, most preferably from about 3 to about 4.5% by weight of hydrogen peroxide (calculated as about 100% H₂O₂) as an oxidizing agent (C).

In a further preferred embodiment, the agents as contemplated herein contain sodium percarbonate as an oxidizing agent (C). Sodium percarbonate (2 Na₂CO₃.3 H₂O₂) is an addition product or adduct of hydrogen peroxide (H₂O₂) to sodium carbonate (Na₂CO₃). Preferred agents as contemplated herein with sodium percarbonate as an oxidizing agent (C), which are preferably initially anhydrous, release hydrogen peroxide when mixed with water, which effects the coupling process of the oxidation dye precursors with each other and the associated dye formation.

Agents preferred as contemplated herein, based on their weight, contain from about 0.5 to about 90% by weight, preferably from about 5 to about 85% by weight, more preferably from about 15 to about 80% by weight, most preferably from about 50 to about 75% by weight sodium percarbonate as an oxidizing agent (C).

Further preferred agents, based on their weight, contain from about 0.5 to about 90% by weight, preferably from about 5 to about 85% by weight, more preferably from about 15 to about 80% by weight, most preferably from about 50 to about 75% by weight of sodium percarbonate as an oxidizing agent (C) and from 0 to about 10% by weight, preferably from about 0.1 to about 8% by weight, particularly preferably from about 0.5 to about 5% by weight of water.

This specification refers to the content of free water. The content of molecularly bound water or water of crystallization which individual ingredients of this embodiment can have is not taken into account. The water content can be determined, for example, based on ISO 4317 (version 2011-12) by employing Karl Fischer titration.

In a further preferred embodiment, the agents as contemplated herein contain, as an oxidizing agent (C), a combination of hydrogen peroxide and at least one persalt, in particular one or more peroxodisulfate salts and/or peroxomonosulfate salts, such as potassium persulfate, sodium persulfate or ammonium persulfate.

Agents preferred as contemplated herein, based on their weight, contain from about 0.5 to about 12% by weight, preferably from about 0.9 to about 7% by weight, particularly preferably from about 1.5 to about 5% by weight, extremely preferably from about 3 to about 4.5% by weight of hydrogen peroxide (calculated as about 100% H₂O₂) and at least one persalt in a total amount of from about 0.1 to about 20% by weight, preferably from about 1 to about 15% by weight, particularly preferably from about 3 to about 10% by weight, as an oxidizing agent (C).

The above-described agent containing hydrogen peroxide as an oxidizing agent (C) is the ready-to-use agent which already contains both at least one oxidation dye precursor (A) and an oxidizing agent (C) which causes the coupling process of the oxidation dye precursor(s) with each other and the associated dye formation, namely hydrogen peroxide. For the preparation of this ready-to-use coloring agent and initiating the dye formation reaction, a first component K1 which contains the at least one oxidation dye precursor (A) and optionally further oxidation dye precursors (OFV), but is free of dissolved hydrogen peroxide, is usually mixed with a second component K2 containing hydrogen peroxide as an oxidizing agent (C). The ready-to-use coloring agent thus obtained is intended for immediate application to the hair to be dyed. The at least one active substance (B) that is, iminodisuccinic acid and/or at least one of its physiologically compatible salts, can be present in component K1 or in component K2. For the large-scale production of the components of an oxidation coloring agent, it can be preferable to formulate the hydrogen peroxide-containing component K2 in the least complex, standardized composition, that is, the active substance (B) that is, iminodisuccinic acid and/or at least one of its physiologically compatible salts, to provide K1 in the OFV-containing component.

To avoid incompatibilities and to prevent premature, unwanted dye formation, the components K1 (oxidation dye precursor-containing) and K2 (oxidizing agent preparation having dissolved hydrogen peroxide) are always prepared separately and only brought into contact with each other shortly before use. The components (K1) and (K2) are preferably provided in the form of a multi-component packaging unit (kit-of-parts) for the consumer.

A second subject of the present disclosure is therefore a multi-component packaging unit (kit-of-parts) for the oxidative dyeing of keratinic fibers, comprising at least two separately prepared components (K1) and (K2), wherein
the first component (K1), in a cosmetic carrier,
   (A) contains at least one oxidation dye precursor of the structure (I) and/or one of its physiologically compatible salts

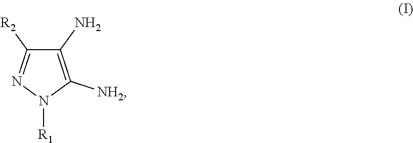

wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, further
(C) optionally at least one oxidizing agent selected from persalts, in particular peroxodisulfate salts and/or peroxomonosulfate salts,
the second component (K2)
   (C) contains hydrogen peroxide dissolved in water,
      wherein at least one of the components (K1) or (K2) (B) contains iminodisuccinic acid and/or at least one of its physiologically compatible salts.

For the large-scale production of the components of an oxidation coloring agent, it can be preferable to formulate the hydrogen peroxide-containing component K2 in the least complex, standardized composition, that is, the active substance (B), that is, iminodisuccinic acid and/or at least one of its physiologically compatible salts, to provide K1 in the OFV-containing component.

As contemplated herein, therefore, a multi-component packaging unit (kit-of-parts) for the oxidative dyeing of keratinic fibers is preferred, comprising at least two separately prepared components (K1) and (K2), wherein
the first component (K1), in a cosmetic carrier,
   (A) contains at least one oxidation dye precursor of structure (I) and/or one of its physiologically compatible salts

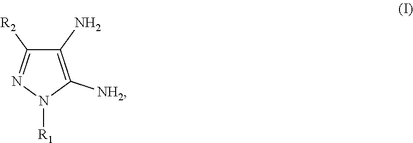

wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, further
(B) iminodisuccinic acid and/or at least one of its physiologically compatible salts, further
(C) optionally contains at least one oxidizing agent selected from persalts, in particular peroxodisulfate salts and/or peroxomonosulfate salts,
the second component (K2)
   (C) contains hydrogen peroxide dissolved in water.

What has been said regarding the preferred oxidation dye precursors (A) of the structure (I), preferred qualitative embodiments of ingredient (B) and preferred qualitative embodiments of ingredient (C) to the first subject of the present disclosure applies mutatis mutandis to the second subject of present disclosure. The amounts of ingredients (A), (B) and (C) preferred as contemplated herein, which are disclosed above for the first subject of the present disclosure, also apply mutatis mutandis to the mixtures of the components (K1) and (K2) of the second subject of the present disclosure. The concentrations of the ingredients (A), (B) and (C) in the components (K1) and (K2) and the mixing ratios of the components (K1) and (K2) to each other are to be selected by the person skilled in the art so that the mixtures of the components (K1) and (K2) have the preferred amounts of ingredients (A), (B) and (C) disclosed above for the first subject of the present disclosure.

A third subject of the present disclosure is therefore a multi-component packaging unit (kit-of-parts) for the oxidative dyeing of keratinic fibers, comprising at least two separately prepared components (K1) and (K2), wherein
the first component (K1), in a cosmetic carrier,
   (A) contains at least one oxidation dye precursor of structure (I) and/or one of its physiologically compatible salts

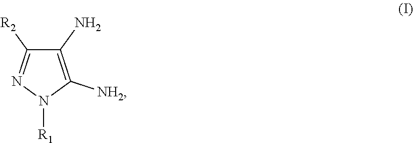

wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, further
(C) contains sodium percarbonate as an oxidizing agent and optionally at least one further oxidizing agent selected from persalts, in particular peroxodisulfate salts and/or peroxomonosulfate salts,
the second component (K2) contains water,
   at least one of the components (K1) or (K2) contains (B) iminodisuccinic acid and/or at least one of its physiologically compatible salts.
   For this third aspect of the present disclosure, it also is preferred that the multi-component packaging unit (kit-of-parts) for the oxidative dyeing of keratinic fibers comprises at least two separately prepared components (K1) and (K2), wherein
the first component (K1), in a cosmetic carrier,
   (A) contains at least one oxidation dye precursor of structure (I) and/or one of its physiologically compatible salts

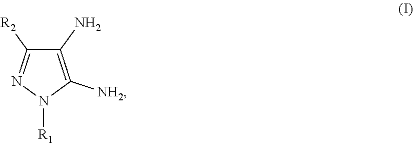

wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, further
(B) iminodisuccinic acid and/or at least one of its physiologically compatible salts and further
(C) contains sodium percarbonate as an oxidizing agent and optionally at least one further oxidizing agent selected from persalts, in particular peroxodisulfate salts and/or peroxomonosulfate salts,
the second component (K2) contains water.

What has been said regarding the preferred oxidation dye precursors (A) of the structure (I), preferred qualitative embodiments of ingredient (B) and preferred qualitative embodiments of ingredient (C) to the first subject of the present disclosure applies mutatis mutandis to the third subject of present disclosure. The amounts of ingredients (A), (B) and (C) preferred, which are disclosed above for the first subject of the present disclosure, also apply mutatis mutandis to the mixtures of the components (K1) and (K2) of the third subject of the present disclosure. The concentrations of the ingredients (A), (B) and (C) in the components (K1) and (K2) and the mixing ratios of the components (K1) and (K2) to each other are to be selected by the person skilled in the art so that the mixtures of the components (K1) and (K2) have the preferred amounts of ingredients (A), (B) and (C) disclosed above for the first subject of the present disclosure.

A fourth subject of the present disclosure is therefore a multi-component packaging unit (kit-of-parts) for the oxidative dyeing of keratinic fibers, comprising at least three separately prepared components (K1), (K2) and (K3), wherein
the first component (K1), in a cosmetic carrier,
   (A) contains at least one oxidation dye precursor of structure (I) and/or one of its physiologically compatible salts

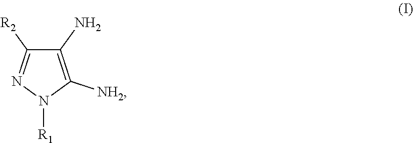

wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, further
(C) optionally contains at least one oxidizing agent selected from persalts, in particular peroxodisulfate salts and/or peroxomonosulfate salts,
contains the second component (K2)
   (C) hydrogen peroxide dissolved in water,
the third component (K3)
   (B) contains iminodisuccinic acid and/or at least one of its physiologically compatible salts.

The fourth subject of the present disclosure presented above provides that component (B), that is, iminodisuccinic acid and/or at least one of its physiologically compatible salts, is present in a third component (K3) separate from the OFV-containing component (K1) and separate from the hydrogen peroxide-containing component (K2). The ready-to-use agent according to this embodiment is obtained by mixing the components (K1), (K2) and (K3) with one another. In principle, the third component (K3) can contain the active substance (B), that is, at least one physiologically compatible salt of iminodisuccinic acid, in pure, undiluted form. This is easily possible, for example, when using the preferred salt tetrasodium iminodisuccinate, since this salt is present as a crystalline solid.

Since the present disclosure essentially relates to a consumer product, it should be designed so that a homogeneous mixture can be prepared from the components (K1), (K2) and (K3) as quickly as possible and with little expenditure on equipment, for immediate application to the suitable for dyeing fibers. Therefore, component (K3) preferably includes an aqueous solution of the active substance (B) used as contemplated herein, that is, iminodisuccinic acid and/or at least one of its physiologically compatible salts, wherein this solution can optionally contain further ingredients.

What has been said regarding the preferred oxidation dye precursors (A) of the structure (I), preferred qualitative embodiments of ingredient (B) and preferred qualitative embodiments of ingredient (C) to the first subject of the present disclosure applies mutatis mutandis to the fourth subject of present disclosure. The amounts of ingredients (A), (B) and (C) preferred as contemplated herein, which are disclosed above for the first subject of the present disclosure, also apply mutatis mutandis to the mixtures of the components (K1), (K2) and (K3) of the fourth subject of the present disclosure. The concentrations of the ingredients (A), (B) and (C) in the components (K1), (K2) and (K3) and the mixing ratios of the components (K1), (K2) and (K3) to each other are to be selected by the person skilled in the art so that the mixtures of the components (K1), (K2) and (K3) have the preferred amounts of ingredients (A), (B) and (C) disclosed above for the first subject of the present disclosure.

A fifth subject of the present disclosure is therefore a multi-component packaging unit (kit-of-parts) for the oxidative dyeing of keratinic fibers, comprising at least three separately prepared components (K1), (K2) and (K3), wherein
the first component (K1), in a cosmetic carrier,
   (A) contains at least one oxidation dye precursor of structure (I) and/or one of its physiologically compatible salts

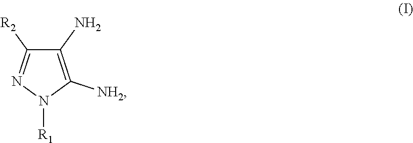

wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, further
(C) contains sodium percarbonate as oxidizing agent and optionally at least one further oxidizing agent selected from persalts, in particular peroxodisulfate salts and/or peroxomonosulfate salts,
the second component (K2) contains water,
the third component (K3)
   (B) contains iminodisuccinic acid and/or at least one of its physiologically compatible salts.

The fifth subject of the present disclosure presented above provides that component (B), that is, iminodisuccinic acid and/or at least one of its physiologically tolerated salts, is present in a third component (K3) separated from the OFV and sodium percarbonate-containing component (K1) and separated from the water-containing component (K2). The ready-to-use agent according to this embodiment is obtained by mixing the components (K1), (K2) and (K3) with one another. In principle, the third component (K3) can contain the active substance (B), that is, at least one physiologically compatible salt of iminodisuccinic acid, in pure, undiluted form. Since the present disclosure essentially relates to a consumer product, it should be designed so that a homogeneous mixture can be prepared from the components (K1), (K2) and (K3) as quickly as possible and with little expenditure on equipment, for immediate application to the suitable for dyeing fibers. Therefore, component (K3) preferably includes an aqueous solution of the active substance (B) used as contemplated herein, that is, iminodisuccinic acid and/or at least one of its physiologically compatible salts, wherein this solution can optionally contain further ingredients.

What has been said regarding the preferred oxidation dye precursors (A) of the structure (I), preferred qualitative embodiments of ingredient (B) and preferred qualitative embodiments of ingredient (C) to the first subject of the present disclosure applies mutatis mutandis to the fifth subject of present disclosure. The amounts of ingredients (A), (B) and (C) preferred as contemplated herein, which are disclosed above for the first subject of the present disclosure, also apply mutatis mutandis to the mixtures of the components (K1), (K2) and (K3) of the firth subject of the present disclosure. The concentrations of the ingredients (A), (B) and (C) in the components (K1), (K2) and (K3) and the mixing ratios of the components (K1), (K2) and (K3) to each other are to be selected by the person skilled in the art so that the mixtures of the components (K1), (K2) and (K3) have the preferred amounts of ingredients (A), (B) and (C) disclosed above for the first subject of the present disclosure.

The first component of the second, third, fourth and fifth subject of the present disclosure is the (preferably alkaline) dyeing preparation (K1) containing at least one oxidation dye precursor (A) of the structure (I) and optionally additional oxidation dye precursors and/or further direct acting dyes.

This dyeing preparation is mixed with an oxidizing agent preparation (K2) before use. For reasons of stability, the oxidizing agent preparation (K2) is preferably adjusted to an acidic pH value and contains the oxidizing agent. The oxidizing agent preparation (K2) of the first, second and fourth subject of the present disclosure is hydrogen peroxide used in the form of an aqueous solution.

The components (K1) and (K2) can be mixed together in different weight ratios (K1)/(K2) of, for example, from about 0.3 to about 3.0, preferably from about 0.5 to about 2.5, particularly preferably from about 0.45 to about 1.5 and most preferably in a weight ratio of 1:1.

A particularly preferred method for oxidative hair dyeing is therefore exemplified in that the first component (K1) and the second component (K2) are mixed together in a weight ratio (K1)/(K2) of from about 0.3 to about 3.0, preferably from about 0.45 to about 2.5, more preferably from about 0.45 to about 1.5 and most preferably in a weight ratio of about 1:1.

The cosmetic carrier for component (K1), which contains the at least one oxidation dye precursor (A) with 4,5-diaminopyrazole as the basic structural element according to the structural formula (I) until the ready-to-use coloring agent is prepared, can be used as a water-based emulsion, as a spray, be formulated as a cream, gel, lotion, paste or shampoo.

Further preferred oxidation coloring agents as contemplated herein contain at least one linear saturated alkanol having 12-30 carbon atoms. In the context of the present disclosure, alkanols having at least 8 carbon atoms are considered as fatty substances, not as surfactants.

Preferred linear saturated alkanols having 12-30 carbon atoms, in particular 16-22 carbon atoms, are selected from cetyl alcohol, stearyl alcohol, arachidyl alcohol, behenyl alcohol and lanolin alcohol and mixtures of these alkanols. Alkanol mixtures particularly preferred as contemplated herein are those obtainable in the industrial hydrogenation of vegetable and animal fatty acids. The total amount of at least one linear saturated alkanol having 12-30 carbon atoms in the oxidation coloring agent as contemplated herein is preferably from about 0.1-20% by weight, preferably from about 0.5-16.5% by weight and more preferably from about 3-10% by weight, in each case based on the weight of the ready-to-use oxidation coloring agent. Further preferably, the total amount of at least one linear saturated alkanol having 12-30 carbon atoms in the OFV-containing component (K1) of the oxidation coloring agent as contemplated herein is from about 0.1-20% by weight, preferably from about 0.5-16.5% by weight, and particularly preferably from about 3-10% by weight, in each case based on the weight of component (K1) of the oxidation coloring agent as contemplated herein.

Further preferred oxidation coloring agents as contemplated herein comprise at least one surfactant or one emulsifier.

Surfactants and emulsifiers in the context of the present application are amphiphilic (bifunctional) compounds which include at least one hydrophobic and at least one hydrophilic molecule part. The hydrophobic radical is preferably a hydrocarbon chain having 8-28 carbon atoms, which can be saturated or unsaturated, linear or branched. Particularly preferably, this C₈-C₂₈ alkyl chain is linear. Basic properties of the surfactants and emulsifiers are the oriented absorption at interfaces and the aggregation to micelles and the formation of lyotropic phases.

When selecting suitable surfactants as contemplated herein, it can be preferable to use a mixture of surfactants in order to optimally adjust the stability of the oxidation coloring agents as contemplated herein.

The total amount of at least one surfactant in the oxidation coloring agents as contemplated herein is preferably from about 0.1-20% by weight, preferably from about 0.5-10% by weight and more preferably from about 1.5-5% by weight, in each case based on the weight of the ready-to-use oxidation coloring agent.

Further preferably, the total amount of at least one surfactant in the OFV-containing component (K1) of the oxidation coloring agent as contemplated herein is from about 0.1-20% by weight, preferably from about 0.5-10% by weight and particularly preferably from about 1.5-5% by weight, in each case based on the weight of component (K1) of the oxidation of the present disclosure.

Preferred surfactants and emulsifiers are selected from anionic, cationic, zwitterionic, amphoteric and nonionic surfactants and emulsifiers and mixtures thereof. These substances are described below.

Preferred oxidation coloring agents as contemplated herein are exemplified in that the at least one surfactant present is selected from nonionic surfactants and anionic surfactants and mixtures thereof. Further preferred oxidation coloring agents as contemplated herein are exemplified in that their OFV-containing component (K1) contains at least one surfactant selected from nonionic surfactants and anionic surfactants and mixtures thereof.

Suitable anionic surfactants are all anionic surfactants suitable for use on the human body which have a water-solubilizing anionic group, for example a carboxylate, sulfate, sulfonate or phosphate group, and a lipophilic alkyl group of about 8 to 30 C atoms, preferably 8 to 24 carbon atoms in the molecule. In addition, glycol or polyglycol ether groups, ester, ether and amide groups and hydroxyl groups can be present in the molecule. Examples of suitable anionic surfactants are, in each case in the form of the sodium, potassium and ammonium and the mono-, di- and trialkanolammonium salts having 2 to 4 C atoms in the alkanol group, linear and branched fatty acids having 8 to 30 C atoms (soaps), polyethoxylated ether carboxylic acids, acyl sarcosides, acyl taurides, acyl isethionates, sulfosuccinic mono- and dialkyl esters and sulfosuccinic monoalkylpolyoxyethyl esters having 1 to 6 ethylene oxide groups, linear alkanesulfonates, linear alpha-olefinsulfonates, sulfonates of unsaturated fatty acids having up to 6 double bonds, alpha-sulfofatty acid methyl esters of fatty acids, C₈-C₂₀ alkyl sulfates and C₈-C₂₀ alkyl ether sulfates having up to 15 oxyethyl groups, mixtures of surface active hydroxysulfonates, sulfated hydroxyalkylpolyethylene and/or hydroxyalkylene glycol ethers, esters of tartaric or citric acid with ethoxylated or propoxylated fatty alcohols, optionally polyethoxylated alkyl and/or alkenyl ether phosphates, sulfated fatty acid alkylene glycol esters, and monoglyceride sulfates and monoglyceride ether sulfates. Preferred anionic surfactants are soaps, fatty acids, C₈-C₂₀ alkyl sulfates, C₈-C₂₀ alkyl ether sulfates and C₈-C₂₀ ether carboxylic acids having 8 to 20 carbon atoms in the alkyl group and up to 12 ethylene oxide groups in the molecule. Particularly preferred is sodium cetearyl sulfate.

The total amount of at least one anionic surfactant in the oxidation coloring agents as contemplated herein is preferably from about 0.01-10% by weight, preferably from about 0.1-5% by weight and more preferably from about 1-3% by weight, in each case based on the weight of the ready-to-use oxidation coloring agent.

Further preferably, the total amount of at least one anionic surfactant in the OFV-containing component (K1) of the oxidation coloring agent as contemplated herein is from about 0.01-10% by weight, preferably from about 0.1-5% by weight and particularly preferably from about 1-3% by weight, in each case based on the weight of component (K1) of the oxidation coloring agent as contemplated herein.

Particularly preferred nonionic surfactants used are selected from with 20 to 100 moles of ethylene oxide per mole of ethoxylated castor oil, ethoxylated C₈-C₂₄ alkanols with 1-200 moles of ethylene oxide per mole, ethoxylated C₈-C₂₄ carboxylic acids with 1-200 moles of ethylene oxide per mole, with 4-80 moles of ethylene oxide per mole of ethoxylated sorbitan monoesters of linear saturated and unsaturated C₁₂-C₃₀ carboxylic acids which can be hydroxylated, in particular those of myristic acid, palmitic acid, stearic acid or mixtures of these fatty acids, alkylmono- and oligoglycosides having 8 to 22 carbon atoms in the alkyl radical and their ethoxylated analogs, and mixtures of the aforementioned substances.

The ethoxylated C₈-C₂₄ alkanols have the formula R¹O(CH₂CH₂O)ₙH, where R¹ stands for a linear or branched alkyl and/or alkenyl radical having 8-24 carbon atoms and n, the average number of ethylene oxide units per molecule, for numbers from 1-200, preferably 2-150, more preferably 4-100, particularly preferably 10-50, even more preferably 12-30 or 20 moles of ethylene oxide to 1 mole of caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol, and technical mixtures thereof. Adducts of 1-200 moles of ethylene oxide with technical fatty alcohols having 12-18 carbon atoms, such as coconut, palm, palm kernel or tallow fatty alcohol, are suitable. Particularly preferred are Laureth-2, Laureth-4, Laureth-10, Laureth-12, Laureth-15, Laureth-20, Laureth-30, Myreth-2, Myreth-4, Myreth-10, Myreth-12, Myreth-15, Myreth-20, Myreth-30, Ceteth-2, Ceteth-4, Ceteth-10, Ceteth-12, Ceteth-15, Ceteth-20, Ceteth-30, Ceteth-50, Ceteth-100, Ceteth-150, Steareth-2, Steareth-4, Steareth-10, Steareth-12, Steareth-15, Steareth-20, Steareth-30, Steareth-50, Steareth-100, Steareth-150, Oleth-2, Oleth-4, Oleth-10, Oleth-12, Oleth-15, Oleth-20, Oleth-30, Ceteareth-2, Ceteareth-4, Ceteareth-10, Ceteareth-15, Ceteareth-12, Ceteareth-15, Ceteareth-20, Ceteareth-30, Ceteareth-50, Ceteareth-100, Ceteareth-150 und Coceth-2, Coceth-4, Coceth-10, Coceth-12, Coceth-15, Coceth-20, Coceth-30, Coceth-50 and Coceth-100.

The ethoxylated C₈-C₂₄ carboxylic acids have the formula R¹O(CH₂CH₂O)ₙH, wherein R¹O stands for a linear or branched saturated or unsaturated acyl radical having 8-24 carbon atoms and n, the average number of ethylene oxide units per molecule, for numbers from 1-200, preferably 10-50, moles of ethylene oxide to 1 mole of caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselic acid, arachic acid, gadoleic acid, behenic acid, erucic acid and brassidic acid, and technical mixtures thereof. Adducts of 1-200, preferably 10 to 50, moles of ethylene oxide to technical fatty acids having 12-18 carbon atoms, such as coconut, palm, palm kernel or tallow fatty acid, are also suitable. Particularly preferred are PEG-50 monostearate, PEG-100 monostearate, PEG-50 monooleate, PEG-100 monooleate, PEG-50 monolaurate and PEG-100 monolaurate.

Preferred with 4-80 moles of ethylene oxide per mole of ethoxylated sorbitan monoesters of linear saturated and unsaturated C₁₂-C₃₀ carboxylic acids which can be hydroxylated are selected from Polysorbate-20, Polysorbate-40, Polysorbate-60 and Polysorbate-80.

Further preferred nonionic surfactants are selected from C₈-C₂₂ alkyl mono- and oligoglycosides. C₈-C₂₂ alkyl mono- and -oligoglycosides constitute known, commercially available surfactants and emulsifiers. They are prepared in particular by reacting glucose or oligosaccharides with primary alcohols having 8-22 carbon atoms. With regard to the glycoside radical, both monoglycosides in which a cyclic sugar radical is glycosidically bound to the fatty alcohol and oligomeric glycosides having a degree of oligomerization of up to about 8, preferably 1-2, are suitable. The degree of oligomerization is a statistical mean value, which is based on a homolog distribution typical for such technical products. Products obtainable under the trademark Plantacare® contain a glucosidically bonded C₈-C₁₆ alkyl group on an oligoglucoside radical whose average degree of oligomerization is at 1-2, in particular 1.2-1.4. Particularly preferred C₈-C₂₂ alkyl mono- and oligoglycosides are selected from octyl glucoside, decyl glucoside, lauryl glucoside, palmityl glucoside, isostearyl glucoside, stearyl glucoside, arachidyl glucoside and behenyl glucoside and mixtures thereof.

Acylglucamides derived from glucamine are also useful as non-ionic oil-in-water emulsifiers.

Further nonionic surfactants as contemplated herein contain as hydrophilic group, for example, a polyol group, a polyalkylene glycol ether group or a combination of polyol and polyglycol ether groups. Such compounds are, for example,
polyglycerol esters and ethoxylated polyglycerol esters of C₈-C₃₀ fatty acids, such as poly (3) glycerol diisostearate (commercial product: Lameform®TGI (BASF)) and poly (2) glycerol polyhydroxystearate (commercial product: Dehymuls®®PGPH (BASF)),
ethoxylated mono-, di- and triesters of glycerol with C₈-C₃₀ fatty acids, such as glycerol monolaurate+20 ethylene oxide and glycerol monostearate+20 ethylene oxide, PEG-x Castor Oil with degree of ethoxylation x=1-80 or PEG-x Hydrogenated Castor Oil with degree of ethoxylation x=1-80,
amine oxides of C₈-C₃₀ fatty amines,
sugar fatty acid esters and addition products of ethylene oxide to sugar fatty acid esters, for example, sucrose stearate, methyl glucose sesquistearate, PEG-20-methyl glucose sesquistearate or PEG-120 methyl glucose dioleate,
addition products of ethylene oxide to fatty acid alkanolamides and fatty amines,
fatty acid N-alkyl glucamides,
monoesters of C₈-C₃₀ fatty acids and ethylene glycol, and
monoesters and diesters of C₈-C₃₀ fatty acids and glycerol, for example, glycerol monostearate or glyceryl distearate.

Oxidation coloring agents preferred as contemplated herein contain at least one nonionic surfactant selected from 20 to 100 moles of ethylene oxide per mole of ethoxylated castor oil, ethoxylated C₈-C₂₄ alkanols with 1-200 moles of ethylene oxide per mole, ethoxylated C₈-C₂₄ carboxylic acids with 1-200 moles of ethylene oxide per mole, with 4-80 moles of ethylene oxide per mole of ethoxylated sorbitan monoesters of linear saturated and unsaturated C₁₂-C₃₀ carboxylic acids which can be hydroxylated, in particular those of myristic acid, palmitic acid, stearic acid or of mixtures of these fatty acids, alkyl mono- and oligoglycosides having 8 to 22 carbon atoms in the alkyl radical and their ethoxylated analogs, acylglucamides derived from glucamine, polyglycerol esters and ethoxylated polyglycerol esters of C8-C30 fatty acids, ethoxylated mono-, di- and triesters of glycerol with C8-C30 fatty acids, amine oxides of C₈-C₃₀ fat amines, sugar fatty acid esters and addition products of ethylene oxide to sugar fatty acid esters, addition products of ethylene oxide to fatty acid alkanolamides and fatty amines, fatty acid N-alkylglucamides, monoesters of C₈-C₃₀ fatty acids and ethylene glycol, monoesters and diesters of C₈-C₃₀ fatty acids and glycerol, and mixtures of the aforementioned substances.

The total amount of at least one nonionic surfactant in the oxidation coloring agents as contemplated herein is preferably from about 0.01-15% by weight, preferably from about 0.1-10% by weight and more preferably from about 1-6% by weight, in each case based on the weight of the ready-to-use oxidation coloring agent.

In another preferred embodiment, the total amount of at least one nonionic surfactant in the OFV-containing component (K1) of the oxidation coloring agent as contemplated herein is from about 0.01-15% by weight, preferably from about 0.1-10% by weight and particularly preferably from about 1-6% by weight, in each case based on the weight of component (K1) of the oxidation coloring agent as contemplated herein.

In another preferred embodiment, the total amount of at least one nonionic surfactant in the oxidizing agent-containing component (K2) of the oxidation coloring agent as contemplated herein is from about 0.01-15% by weight, preferably from about 0.1-10% by weight and particularly preferably from about 1-4% by weight, in each case based on the weight of component (K2) of the oxidation coloring agent as contemplated herein.

In another preferred embodiment, the oxidation coloring agent as contemplated herein contains a total of from about 0.1 to about 15% by weight, preferably from about 0.5 to about 10% by weight and particularly preferably from about 1-5% by weight, of a mixture of nonionic and anionic surfactants, in each case based on the weight of the ready-to-use oxidation coloring agent.

In another preferred embodiment, the OFV-containing component (K1) of the oxidation coloring agent contains a total of from about 0.1 to about 15% by weight, preferably from about 0.5 to about 10% by weight and particularly preferably from about 1-5% by weight, of a mixture of nonionic and anionic surfactants, in each case based on the weight of component (K1).

In another preferred embodiment, the oxidizing agent-containing component (K2) of the oxidation coloring agent contains a total of from about 0.1 to about 15% by weight, preferably from about 0.5 to about 10% by weight and particularly preferably from about 1-5% by weight, of a mixture of nonionic and anionic surfactants, in each case based on the weight of component (K2).

Zwitterionic surfactants are surface-active compounds which carry at least one quaternary ammonium group and at least one carboxylate, sulfonate or sulfate group in the molecule. Particularly suitable zwitterionic surfactants are the so-called betaines such as N-alkyl-N,N-dimethylammonium glycinates, for example, cocoalkyl dimethylammonium glycinate, N-acylaminopropyl N,N-dimethylammonium glycinates, for example, cocoacylaminopropyl-dimethylammonium glycinate, and 2-alkyl-3-carboxymethyl-3-hydroxyethyl-imidazolines each having 8 to 18 C atoms in the alkyl or acyl group and cocoacylamino ethyl hydroxyethyl carboxymethyl glycine. A preferred zwitterionic surfactant is the fatty acid amide derivative known by the INCI name Cocamidopropyl Betaine.

Amphoteric surfactants are understood to mean those surface-active compounds which, apart from a C₈-C₂₄ alkyl or acyl group in the molecule, contain at least one free amino group and at least one —COOH or —SO₃H group and which are capable of forming internal salts. Examples of suitable amphoteric surfactants are N-alkylglycines, N-alkylpropionic acids, N-alkylaminobutyric acids, N-alkyliminodipropionic acids, N-hydroxyethyl-N-alkylamidopropylglycine, N-alkyltaurines, N-alkylsarcosines, 2-alkylaminopropionic acids and alkylaminoacetic acids, each having 8 to 24 C atoms in the alkyl group. Particularly preferred amphoteric surfactants are N-coco alkylamine propionate and C₁₂-C₁₈ acylsarcosine.

In a further preferred embodiment, the oxidation coloring agent as contemplated herein contains a total of from about 0.1 to about 10% by weight, preferably from about 0.2 to about 6% by weight and particularly preferably from about 1-2% by weight, of at least one zwitterionic and/or amphoteric surfactant, in each case based on the weight of the ready oxidation coloring agent.

In another preferred embodiment, the OFV-containing component (K1) of the oxidation coloring agent as contemplated herein contains a total of from about 0.1 to about 10% by weight, preferably from about 0.2 to about 6% by weight and particularly preferably from about 1-4% by weight, of at least one zwitterionic or/and an amphoteric surfactant, in each case based on the weight of component (K1).

Optionally, the oxidation coloring agent as contemplated herein, based on its weight, contains at least one cosmetic oil in a total amount of from about 0.01-10% by weight, preferably from about 0.1-5% by weight, particularly preferably from about 0.5-4% by weight, most preferably from about 1-2% by weight. In another preferred embodiment, the OFV-containing component (K1) of the oxidation coloring agent contains a total of from about 0.1 to about 10% by weight, preferably from about 0.2 to about 6% by weight and particularly preferably from about 1-4% by weight, of at least one cosmetic oil, in each case based on the weight of component (K1). In another preferred embodiment, the oxidizing agent-containing component (K2) of the oxidation coloring agent contains a total of from about 0.1 to about 10% by weight, preferably from about 0.2 to about 6% by weight and particularly preferably from about 1-4% by weight, of at least one cosmetic oil, in each case based on the weight of component (K2). The cosmetic oil is liquid under normal conditions (20° C., 1013.25 mbar); essential oils and perfume oils or fragrances are not counted among the cosmetic oils. The liquid cosmetic oils are immiscible with water under normal conditions. Essential oils as contemplated herein are understood to mean mixtures of volatile components which are prepared by steam distillation from vegetable raw materials, such as citrus oils. As far as a cosmetic oil is mentioned in the present application, this is always a cosmetic oil that is not a fragrance and not an essential oil, is liquid under normal conditions and immiscible with water.

The definition of a fragrance in the context of the present application is in accordance with the conventional expert definition as it can be found in the RÖMPP Chemie Lexikon, as of December 2007. Thereafter, a fragrance is a chemical compound having odor and/or taste that excites the hair cell receptors of the olfactory system (adequate stimulus). The physical and chemical properties necessary for this are a low molecular mass of at most about 300 g/mol, a high vapor pressure, minimal water and high lipid solubility and weak polarity and the presence of at least one osmophoric group in the molecule. In order to delineate volatile, low molecular weight substances which are usually and also not regarded and used as fragrances, but primarily as solvents, for example ethanol, propanol, isopropanol and acetone, fragrances as contemplated herein have a molecular mass of from about 74 to about 300 g/mol, contain at least one osmophoric group in the molecule and have a smell and/or taste, that is, they excite the receptors of the hair cells of the olfactory system.

Further particularly preferred cosmetic oils as contemplated herein are selected from the esters of linear or branched saturated or unsaturated fatty alcohols having 2-30 carbon atoms with linear or branched saturated or unsaturated fatty acids having 2-30 carbon atoms which can be hydroxylated. These include cetyl 2-ethylhexanoate, 2-hexyldecyl stearate, 2-hexyldecyl laurate, isodecyl neopentanoate, isononyl isononanoate, 2-ethylhexyl palmitate and 2-ethylhexyl stearate. Also preferred are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate.

Further preferred oils are selected from natural and synthetic hydrocarbons, more preferably from mineral oils, paraffin oils, C₁₈-C₃₀ isoparaffins, in particular isoeicosane, polyisobutenes and polydecenes, for example, which are available, for example, under the name Emery® 3004, 3006, 3010 or under the name Ethylflo® from Albemarle or Nexbase® 2004G from Nestle, further selected from C₈-C₁₆ isoparaffins, in particular from isodecane, isododecane, isotetradecane and isohexadecane and mixtures thereof, and 1,3-di-(2-ethylhexyl)-cyclohexane.

Further preferred oils as contemplated herein are selected from the benzoic acid esters of linear or branched C8-22 alkanols. Especially preferred are benzoic C12-C15 alkyl esters, benzoic acid isostearyl ester, ethylhexyl benzoate and benzoic acid octyldodecyl ester.

Further preferred oils are selected from fatty alcohols having 6-30 carbon atoms which are unsaturated or branched and saturated or branched and unsaturated. The branched alcohols are also often referred to as Guerbet alcohols, since they are obtainable by the Guerbet reaction. Preferred alcohol oils are 2-hexyldecanol, 2-octyldodecanol, 2-ethylhexyl alcohol and isostearyl alcohol.

Further preferred oils are selected from mixtures of Guerbet alcohols and Guerbet alcohol esters, for example, mixtures of 2-hexyldecanol and 2-hexyldecyl laurate.

Further preferred cosmetic oils as contemplated herein are selected from the triglycerides (=triple esters of glycerol) of linear or branched, saturated or unsaturated, optionally hydroxylated C8-30 fatty acids. Particularly preferred can be the use of natural oils, for example, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderflower seed oil, currant seed oil, jojoba oil, linseed oil, macadamia nut oil, corn oil, almond oil, Marula oil, evening primrose oil, olive oil, palm oil, palm kernel oil, Brazil nut oil, pecan oil, peach kernel oil, rapeseed oil, castor oil, sea buckthorn fruit oil, sea buckthom seed oil, sesame oil, soybean oil, sunflower oil, grapeseed oil, walnut oil, wild rose oil, wheat germ oil, and the liquid portions of coconut oil and the like. However, synthetic triglyceride oils are also preferred, in particular capric/caprylic triglycerides, for example, the commercial products Myritol® 318, Myritol® 331 (BASF) with unbranched fatty acid radicals and glyceryl triisostearin with branched fatty acid radicals.

Further preferred cosmetic oils as contemplated herein are selected from the dicarboxylic acid esters of linear or branched C2-C10 alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further preferred cosmetic oils as contemplated herein are selected from the addition products of 1 to 5 propylene oxide units to mono- or multivalent C8-C22 alkanols such as octanol, decanol, decanediol, lauryl alcohol, myristyl alcohol and stearyl alcohol, for example, PPG-2 myristyl ether and PPG-3 myristyl ether.

Further preferred cosmetic oils are selected from the addition products of at least 6 ethylene oxide and/or propylene oxide units of monovalent or polyvalent C3-22 alkanols such as glycerol, butanol, butanediol, myristyl alcohol and stearyl alcohol, which can be esterified if desired, for example, PPG-14 butyl ether, PPG-9-butyl ether, PPG-10-butanediol, PPG-15 stearyl ether and glycereth-7-diisononanoate.

Further preferred cosmetic oils as contemplated herein are selected from the C8-C22 fatty alcohol esters of monovalent or multivalent C2-C7 hydroxycarboxylic acids, in particular the esters of glycolic acid, lactic acid, malic acid, tartaric acid, citric acid and salicylic acid.

Further preferred cosmetic oils are selected from the symmetrical, asymmetric or cyclic esters of carbonic acid with C₃₋₂₂ alkanols, C₃₋₂₂ alkanediols or C₃₋₂₂ alkanetriols, for example, dicaprylyl carbonate, or the esters according to the teachings of DE 19756454 A1, in particular glycerol carbonate.

Further cosmetic oils which can be preferred are selected from the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols.

Further cosmetic oils which are suitable as contemplated herein are selected from silicone oils including, for example, dialkyl and alkylaryl siloxanes such as cyclopentasiloxane, cyclohexasiloxane, dimethylpolysiloxane and methylphenylpolysiloxane, but also hexamethyldisiloxane, octamethyltrisiloxane and decamethyltetrasiloxane. Preferred can be volatile silicone oils, which can be cyclic, such as octamethylcyclotetrasiloxane, decamethylcyclopentasiloxane and dodecamethylcyclohexasiloxane, and mixtures thereof, such as those contained in commercial products DC 244, 245, 344 and 345 from Dow Corning. Likewise suitable are volatile linear silicone oils, in particular hexamethyldisiloxane (L2), octamethyltrisiloxane (L3), decamethyltetrasiloxane (L4) and any mixtures of two or three of L2, L3 and/or L4, preferably mixtures as are available, for example, in the commercial products DC 2-1184, Dow Corning® 200 (0.65 cSt) and Dow Corning® 200 (1.5 cSt) from Dow Corning. Preferred nonvolatile silicone oils are selected from higher molecular weight linear dimethylpolysiloxanes, commercially available, for example, under the name Dow Corning® 190, Dow Corning® 200 Fluid having kinematic viscosities (about 25° C.) in the range of from about 5-100 cSt, preferably from about 5-50 cSt or also from about 5-10 cSt, and dimethylpolysiloxane having a kinematic viscosity about (25° C.) of about 350 cSt.

It can be extremely preferable as contemplated herein to use mixtures of the aforementioned oils.

Preferred coloring agents as contemplated herein are exemplified in that the cosmetic oil is selected from natural and synthetic hydrocarbons, more preferably from paraffin oils, C₁₈-C₃₀ isoparaffins, in particular isoeicosane, polyisobutenes and polydecenes, C₈-C₁₆isoparaffins, and 1,3-di-(2-ethylhexyl) cyclohexane; the benzoic acid esters of linear or branched C₈₋₂₂ alkanols; fatty alcohols having 6-30 carbon atoms which are unsaturated or branched and saturated or branched and unsaturated; triglycerides of linear or branched, saturated or unsaturated, optionally hydroxylated C₈₋₃₀ fatty acids, in particular natural oils; the dicarboxylic acid esters of linear or branched C₂-C₁₀ alkanols; the esters of linear or branched saturated or unsaturated fatty alcohols having 2-30 carbon atoms with linear or branched saturated or unsaturated fatty acids having 2-30 carbon atoms which can be hydroxylated; the addition products of 1 to 5 propylene oxide units of monovalent or polyvalent C₈₋₂₂ alkanols; the addition products of at least 6 ethylene oxide and/or propylene oxide units on monovalent or polyvalent C₃₋₂₂ alkanols; the C_{8-C22} fatty alcohol esters of monovalent or polyvalent C₂-C₇ hydroxycarboxylic acids; the symmetrical, asymmetrical or cyclic esters of carbonic acid with C₃₋₂₂ alkanols, C₃₋₂₂ alkanediols or C₃₋₂₂ alkanetriols; the esters of dimers of unsaturated C₁₂-C₂₂ fatty acids (dimer fatty acids) with monovalent linear, branched or cyclic C₂-C₁₈ alkanols or with polyvalent linear or branched C₂-C₆alkanols; silicone oils and mixtures of the aforementioned substances.

In addition, the multi-component packaging units as contemplated herein can also contain one or more further separately prepared components. This or these additional separately prepared components can, for example, be a pretreatment agent or an aftertreatment agent, such as shampoos or conditioners.

The ready-to-use oxidative coloring agent is preferably adjusted to an alkaline pH for sufficient swelling of the keratin fibers. Dyeing processes on keratin fibers usually take place in an alkaline medium. However, in order to preserve the keratin fibers and the skin as much as possible, the setting of too high a pH value is not desirable. Therefore, it is preferred that the pH value of the ready-to-use agent is a value from about 8.0 to about 10.5, more preferably from about 8.7 to about 10.3, even more preferably from about 9.0 to about 10.2, and most preferably from about 9.2 to about 10.1. The indicated pH values are values measured at a temperature of about 22° C. using a glass electrode.

The alkalizing agents necessary for the adjustment of the alkaline pH are generally contained together with the at least one oxidation dye precursor in the component (K1). The alkalizing agents which can be used as contemplated herein can be selected from the group of ammonia, alkanolamines, basic amino acids and inorganic alkalizing agents such as (earth) alkali metal hydroxides, (earth) alkali metal metasilicates, (earth) alkaline metal phosphates and (earth) alkali metal hydrogen phosphates. Preferred inorganic alkalizing agents are sodium hydroxide, potassium hydroxide, sodium silicate and sodium metasilicate. The alkanolamines which can be used as alkalizing agents are preferably selected from primary amines having a C₂-C₆ alkyl basic body which carries at least one hydroxyl group. Further suitable alkanolamines are selected from the group of 2-aminoethane-1-ol (monoethanolamine), 3-aminopropan-1-ol, 4-aminobutan-1-ol, 5-aminopentan-1-ol, 1-aminopropane-2 ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Very particularly preferred alkanolamines as contemplated herein are selected from the group of 2-aminoethane-1-ol, 2-amino-2-methylpropan-1-ol and 2-amino-2-methylpropane-1,3-diol. The basic amino acids usable as an alkalizing agent as contemplated herein are preferably selected from the group of arginine, lysine, omithine and histidine, more preferably arginine. However, it has been found in the context of the investigations on the present disclosure that further preferred agents as contemplated herein additionally contain an organic alkalizing agent. An embodiment of the first subject of the present disclosure is exemplified in that the agent additionally contains at least one alkalizing agent which is selected from the group which is formed from ammonia, alkanolamines and basic amino acids, in particular ammonia, monoethanolamine and arginine or its compatible salts. The alkalizing agent(s) are preferably present together with the oxidation dye precursors in the dyeing preparation (K1).

The second component (K2) of the first, second and fourth subject of the present disclosure is an oxidizing agent preparation containing hydrogen peroxide in aqueous solution. The concentration of the hydrogen peroxide in the oxidizing agent preparation (K2) is determined, on the one hand, by the legal requirements and, on the other hand, by the desired effect; preferably from about 6 to about 12% by weight solutions in water are used. Preparations (K2) preferred as contemplated herein contain from about 1 to about 24% by weight, preferably from about 3 to about 12.5% by weight, particularly preferably from about 6 to about 10% by weight and in particular from about 3 to about 6% by weight of hydrogen peroxide, in each case based on the weight of the oxidizing agent preparation (K2).

The weight-related mixing ratios of (K1) to (K2) should be selected accordingly so that the ready-to-use coloring agent, based on its weight, contains from about 0.5 to about 12% by weight, preferably from about 0.9 to about 7% by weight, particularly preferred from about 1.5 to about 5% by weight, most preferably from about 3 to about 4.5% by weight of hydrogen peroxide (calculated as 100% H₂O₂).

With regard to further preferred embodiments of the multi-component packaging unit (kit-of-parts) as contemplated herein, what has been said about the agents as contemplated herein applies mutatis mutandis.

For further fine shading of the desired shade of redness, the agents as contemplated herein can additionally contain one or more further oxidation dyes of the developer type. In particular, good results could be obtained when, in addition to the at least one oxidation dye precursor (A), at least one compound from the group including p-aminophenol, 4-amino-3-methylphenol, 4-amino-2-aminomethylphenol, 4-amino-2-(1,2-dihydroxyethyl) phenol, 4-amino-2-(diethylaminomethyl) phenol, and/or the physiologically compatible salts of these compounds is present. Particularly preferred from this group is the developer component 4-amino-3-methylphenol, also referred to as “oxyrot”.

In a further particularly preferred embodiment, an agent as contemplated herein additionally contains one or more compounds from the group including p-aminophenol, 4-amino-3-methylphenol, 4-amino-2-aminomethylphenol, 4-amino-2-(1,2-dihydroxyethyl) phenol, 4-amino-2-(diethylaminomethyl) phenol, and/or the physiologically compatible salts of these compounds, preferably in a total amount of from about 0.001 to about 0.4% by weight, particularly preferably from about 0.01 to about 0.2% by weight, most preferably from about 0.05 to about 0.1% by weight, in each case based on the ready-to-use coloring agent as contemplated herein.

In a further particularly preferred embodiment, an agent as contemplated herein additionally contains 4-amino-3-methylphenol and/or a physiologically compatible salt of this compound, preferably in a total amount of from about 0.001 to about 0.4% by weight, particularly preferably from about 0.01 to about 0.2% by weight, most preferably from about 0.05 to about 0.1% by weight, in each case based on the ready-to-use coloring agent as contemplated herein.

In a further particularly preferred embodiment, a kit as contemplated herein is exemplified in that the OFV-containing component (K1) additionally contains one or more compounds from the group including p-aminophenol, 4-amino-3-methylphenol, 4-amino-2 aminomethylphenol, 4-amino-2-(1,2-dihydroxyethyl) phenol, 4-amino-2-(diethylaminomethyl) phenol, and/or the physiologically compatible salts of these compounds, preferably in a total amount of from about 0.002 to about 0.8% by weight, particularly preferably from about 0.02 to about 0.4% by weight, most preferably from about 0.1 to about 0.2% by weight, in each case based on the weight of component (K1).

In a further particularly preferred embodiment, a kit as contemplated herein is exemplified in that the OFV-containing component (K1) additionally contains 4-amino-3-methylphenol and/or a physiologically compatible salt of this compound, preferably in a total amount of from about 0.002 to about 0.8% by weight, particularly preferably from about 0.02 to about 0.4% by weight, most preferably from about 0.1 to about 0.2% by weight, in each case based on the weight of component (K1).

Particular preference is furthermore given to an agent for the oxidative dyeing of keratinic fibers, contained in a cosmetic carrier, based on the total weight of the ready-to-use agent, wherein

(A) 4,5-diamino-1-(2-hydroxyethyl)-1H-pyrazole (structure I-A) is present in a total amount of from about 0.01 to about 2.5% by weight, preferably from about 0.1 to about 1.8% by weight, particularly preferably from about 0.2 to about 1.0% by weight, most preferably from about 0.4 to about 0.9% by weight, wherein the amount specifications are based on the weight of the free 4,5-diaminopyrazole base in relation to the weight of the agent as contemplated herein, further
(B) iminodisuccinic acid and/or at least one of its physiologically compatible salts is/are present in a total amount of from about 0.01 to about 2.5% by weight, preferably from about 0.05 to about 1.5% by weight, particularly preferably from about 0.1 to about 1.0% by weight, exceptionally preferably from about 0.1 to about 0.5% by weight, wherein the amount specifications are based on the weight of free iminodisuccinic acid in relation to the weight of the agent as contemplated herein, further
(C) at least one oxidizing agent other than atmospheric oxygen, and additionally one or more compounds from p-aminophenol, 4-amino-3-methylphenol, 4-amino-2-aminomethylphenol, 4-amino-2-(1,2-dihydroxyethyl) phenol, 4-amino-2-(diethylaminomethyl) phenol, and the physiologically compatible salts of these compounds, in a total amount of from about 0.001 to about 0.4% by weight, particularly preferably from about 0.01 to about 0.2% by weight, most preferably from about 0.05 to about 0.1% by weight, in each case based on the ready-to-use coloring agent as contemplated herein.

Particular preference is given to an agent for the oxidative dyeing of keratinic fibers, contained in a cosmetic carrier, based on the total weight of the ready-to-use agent, 4,5-diamino-1-(2-hydroxyethyl)-1H-pyrazole (structure I-A) is present in a total amount of from about 0.01 to about 2.5% by weight, preferably from about 0.1 to about 1.8% by weight, particularly preferably from about 0.2 to about 1.0% by weight, most preferably from about 0.4 to about 0.9% by weight, wherein the amount specifications are based on the weight of the free 4,5-diaminopyrazole base in relation to the weight of the agent as contemplated herein, further

(B) tetrasodium iminodisuccinate is/are present in a total amount of from about 0.01 to about 2.5% by weight, preferably from about 0.05 to about 1.5% by weight, more preferably from about 0.1 to about 1.0% by weight, most preferably form about 0.1 to about 0.5% by weight, wherein the amount specifications refer to the weight converted to free iminodisuccinic acid in relation to the weight of the agent as contemplated herein, further
(C) at least one oxidizing agent other than atmospheric oxygen, and additionally one or more compounds from the group including p-aminophenol, 4-amino-3-methylphenol, 4-amino-2-aminomethylphenol, 4-amino-2-(1,2-dihydroxyethyl) phenol, 4-amino-2-(diethylaminomethyl) phenol, and/or the physiologically compatible salts of these compounds, in a total amount of from about 0.001 to about 0.4% by weight, particularly preferably from about 0.01 to about 0.2% by weight, most preferably from about 0.05 to about 0.1% by weight, in each case based on the ready-to-use coloring agent as contemplated herein.

Particular preference is furthermore given to an agent for the oxidative dyeing of keratinic fibers, contained in a cosmetic carrier, based on the total weight of the ready-to-use agent,

4,5-diamino-1-(2-hydroxyethyl)-1H-pyrazole (structure I-A) is present in a total amount of from about 0.01 to about 2.5% by weight, preferably from about 0.1 to about 1.8% by weight, particularly preferably from about 0.2 to about 1.0% by weight, most preferably from about 0.4 to about 0.9% by weight, wherein the amount specifications are based on the weight of the free 4,5-diaminopyrazole base in relation to the weight of the agent as contemplated herein, further
(B) tetrasodium iminodisuccinate is/are present in a total amount of from about 0.01 to about 2.5% by weight, preferably from about 0.05 to about 1.5% by weight, more preferably from about 0.1 to about 1.0% by weight, most preferably from about 0.1 to about 0.5% by weight, wherein the amount specifications refer to the weight converted to free iminodisuccinic acid in relation to the weight of the agent as contemplated herein, further
(C) at least one oxidizing agent other than atmospheric oxygen, and additionally 4-amino-3-methylphenol and/or the physiologically tolerated salts of this compound, in a total amount of from about 0.001 to about 0.4% by weight, particularly preferably from about 0.01 to about 0.2% by weight, very preferably from about 0.05 to about 0.1% by weight, in each case based on the ready-to-use coloring agent as contemplated herein.

Further preferred coloring agents as contemplated herein are exemplified in that additional developer-type oxidation dye precursors selected from the group formed of p-phenylenediamine, toluene-2,5-diamine, 2-(2,5-diaminophenyl) ethanol, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl) propyl] amine, N,N′-bis-(2-hydroxyethyl)-N,N′-bis-(4-aminophenyl)-1,3-diamino-propane-2-ol, bis-(2-hydroxy-5-aminophenyl) methane, 1,3-bis-(2,5-diaminophenoxy) propan-2-ol, N,N′-bis-(4-aminophenyl)-1,4-diazacycloheptane, 1,10-bis-(2,5-diaminophenyl)-1,4,7,10-tetraoxadecane, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]-pyrazol-1-one and their physiologically compatible salts, are not present or only in a total amount of at most about 0.08% by weight, preferably at most about 0.02% by weight, in each case based on the weight of the ready-to-use coloring agent.

Preferred physiologically compatible salts of the oxidation dye precursors which have one or more amine groups are in particular the hydrochlorides (monohydrochloride×HCl, or dihydrochloride×2 HCl), the sulfate (x H₂SO₄), and the hydrobromides (monohydrobromide×HBr, or dihydrobromide×2 HBr) of the compound.

Furthermore, the agents can additionally also contain one or more coupler-type oxidation dye precursors. Coupler components do not alone form significant dyeing in the context of oxidative dyeing, but always require the presence of developer components. Coupler components in the context of the present disclosure allow at least one substitution of a chemical radical of the coupler by the oxidized form of the developer component. Covalent bonds form between coupler and developer components.

Preference is given to selecting at least one compound from one of the following classes as a suitable coupler component:
m-aminophenol and/or its derivatives,
m-dihydroxybenzene and/or its derivatives,
m-diaminobenzene and/or its derivatives,
o-diaminobenzene and/or its derivatives,
o-aminophenol derivatives, such as o-aminophenol,
naphthalene derivatives having at least one hydroxy group,
di- or trihydroxybenzene and/or derivatives thereof,
pyridine derivatives,
pyrimidine derivatives,
monohydroxyindole derivatives and/or monoamine indole derivatives,
monohydroxyindoline derivatives and/or monoaminoindoline derivatives,
pyrazolone derivatives such as 1-phenyl-3-methylpyrazol-5-one,
morpholine derivatives, such as 6-hydroxybenzomorpholine or 6-aminobenzomorpholine,
quinoxaline derivatives, such as 6-methyl-1,2,3,4-tetrahydroquinoxaline.
   Mixtures of two or more compounds from one or more of these classes are also within the scope of this embodiment.

A further preferred embodiment is an agent characterized by additionally containing at least one coupler-type oxidation dye precursor selected from 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy) ethanol, 1,3-bis(2,4-diaminophenoxy) propane, 1-methoxy-2-amino-4-(2-hydroxyethylamino) benzene, 1,3-bis(2,4-diaminophenyl) propane, 2,6-bis(2′-hydroxyethylamino)-1-methylbenzene, 2-({3-[(2-hydroxyethyl)amino]-4-methoxy-5-methylphenyl}amino) ethanol, 2-({3-[(2-hydroxyethyl)amino]-2-methoxy-5-methylphenyl}amino) ethanol, 2-({3-[(2-hydroxyethyl)amino]-4,5-dimethylphenyl}amino) ethanol, 2-[3-morpholine-4-ylphenyl)amino] ethanol, 3-amino-4-(2-methoxyethoxy)-5-methylphenylamine, 1-amino-3-bis-(2-hydroxyethyl) aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline and/or 7-hydroxyindoline and their physiological compatible salts.

In principle, the agents as contemplated herein can also contain at least one direct acting dye from the group of anionic, nonionic and/or cationic dyes.

Particular preference is given to one or more nonionic direct acting dyes from the group HC Yellow 2, HC Yellow 4, HC Yellow 5, HC Yellow 6, HC Yellow 12, HC Orange 1, Disperse Orange 3, HC Red 1, HC Red 3, HC Red 7,HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, 1,4-diamino-2-nitrobenzene, 2-amino-4-nitrophenol, 1,4-bis-(2-hydroxyethyl)-amino-2-nitrobenzene, 3-nitro-4-(2-hydroxyethyl) aminophenol, 2-(2-hydroxyethyl) amino-4,6-dinitrophenol, 4-[(2-hydroxyethyl) amino]-3-nitro-1-methylbenzene, 1-amino-4-(2-hydroxyethyl) amino-5-chloro-2-nitrobenzene, 4-amino-3-nitrophenol, 1-(2′-ureidoethyl) amino-4-nitrobenzene, 2-[(4-amino-2-nitrophenyl) amino]benzoic acid, 4-[(3-hydroxypropyl) amino]-3-nitrophenol, 4-nitro-o-phenylenediamine, 6-nitro-1,2,3,4-tetrahydroquinoxaline, 2-hydroxy-1,4-naphthoquinone, picramic acid and its salts, 2 amino-6-chloro-4-nitrophen ol, 4-ethylamino-3-nitrobenzoic acid and 2-chloro-6-ethylamino-4-nitrophenol.

In a further particularly preferred embodiment, the agent as contemplated herein additionally contains one or more nonionic direct acting dyes from the group HC Yellow 2, HC Yellow 4, HC Yellow 5, HC Yellow 6, HC Yellow 12, HC Orange 1, Disperse Orange 3, HC Red 1, HC Red 3, HC Red 7,HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, 1,4-diamino-2-nitrobenzene, 2-amino-4-nitrophenol, 1,4-bis-(2-hydroxyethyl)-amino-2-nitrobenzene, 3-nitro-4-(2-hydroxyethyl) aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino] benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

In addition, anionic direct acting dyes known under the international names or trade names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue and tetrabromophenol blue can be present.

Suitable cationic direct acting dyes are cationic triphenylmethane dyes such as Basic Blue 7, Basic Blue 26, Basic Violet 2 and Basic Violet 14, aromatic systems which are substituted with a quaternary nitrogen group such as Basic Yellow 57, Basic Red 76, Basic Blue 99, Basic Brown 16 and Basic Brown 17, cationic anthraquinone dyes such as HC Blue 16 (Bluequat B) and direct acting dyes which contain a heterocycle having at least one quaternary nitrogen atom, in particular Basic Yellow 87, Basic Orange 31 and Basic Red 51. The cationic direct acting dyes that are sold under the trademark Arianor are also suitable cationic direct acting dyes as contemplated herein.

The additional oxidation dye precursors, that is, developer components which are different from the compounds of group (A), furthermore coupler components and optionally additionally contained direct acting dyes, can, for example, be present in an amount of from about 0.0001 to about 5.0% by weight, preferably from about 0.001 to about 0.5% by weight, in each case based on the total weight of the agent as contemplated herein.

Furthermore, the agents as contemplated herein can contain other active substances, auxiliaries and additives, such as nonionic polymers such as, vinylpyrrolidinone/vinyl acrylate copolymers, polyvinylpyrrolidinone, vinylpyrrolidinone/vinyl acetate copolymers, polyethylene glycols and polysiloxanes; additional silicones, such as volatile or nonvolatile, straight-chain, branched or cyclic, crosslinked or uncrosslinked polyalkylsiloxanes (such as dimethicones or cyclomethicones), polyarylsiloxanes and/or polyalkylarylsiloxanes, in particular polysiloxanes having organofunctional groups, such as substituted or unsubstituted amines (amodimethicones), carboxyl, alkoxy and/or hydroxyl groups (dimethicone copolyols), linear polysiloxane (A)—polyoxyalkylene (B) block copolymers, grafted silicone polymers; cationic polymers such as quaternized cellulose ethers, polysiloxanes having quaternary groups, dimethyldiallylammonium chloride polymers, acrylamide-dimethyldiallyl-ammonium chloride copolymers, with diethyl sulfate quaternized dimethylaminoethylmethacrylate-vinylpyrrolidinone copolymers, vinylpyrrolidinone-imidazolinium methochloride copolymers and quaternized polyvinyl alcohol; zwitterionic and amphoteric polymers; anionic polymers such as polyacrylic acids or crosslinked polyacrylic acids; structurants such as glucose, maleic acid and lactic acid, hair conditioning compounds such as phospholipids, for example, lecithin and cephalins; perfume oils, dimethylisosorbide and cyclodextrins; fiber structure improving agents, in particular mono-, di- and oligosaccharides such as glucose, galactose, fructose, fruit sugar and lactose; dyes for staining the agent; anti-dandruff agents such as Piroctone Olamine, Zinc Omadine and Climbazole; amino acids and oligopeptides; protein hydrolyzates on animal and/or vegetable basis, and in the form of their fatty acid condensation products or optionally anionically or cationically modified derivatives; vegetable oils; sunscreens and UV blockers; active substances such as panthenol, pantothenic acid, pantolactone, allantoin, pyrrolidinonecarboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; waxes, such as beeswax, montan wax and paraffin waxes; swelling and penetration substances such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescing agents such as ethylene glycol mono- and distearate and PEG-3-distearate; pigments and propellants such as propane-butane mixtures, N₂O, dimethyl ether, CO₂ and air.

A further subject of the present disclosure is a method for the oxidative hair coloring of keratinic fibers, in particular human hair, in which an agent as contemplated herein or preferred as contemplated herein, in particular an agent according to any one of claims 1 to 7, is applied to the fibers, in particular the hair, remains there on the fibers for a period of from about 1 to about 60 minutes, preferably from about 20 to about 45 minutes, at room temperature and/or at least about 30° C., the fibers, in particular the hair, are then rinsed with water and/or a cleaning composition and optionally an aftertreatment agent is applied to the fibers, in particular the hair, which is optionally rinsed, and the fibers, in particular the hair, are subsequently dried.

A further subject of the present disclosure is a method for the oxidative hair coloring of keratinic fibers, in particular human hair, in which the components of a kit as contemplated herein or preferred as contemplated herein, in particular a kit according to any one of claims 8 to 11, are applied simultaneously or directly successively without rinsing on the fibers, in particular the hair, remain there on the fibers for a period of about 1 to about 60 minutes, preferably from about 20 to about 45 minutes, at room temperature and/or at least about 30° C., the fibers, in particular the hair, are then rinsed with water and/or a cleaning composition and optionally an aftertreatment agent is applied to the fibers, in particular the hair, which is optionally rinsed, and the fibers, in particular the hair, are subsequently dried.

The term “room temperature” refers to the temperature as contemplated herein in the room in which a person usually uses a hair coloring agent, thus usually a bathroom or a hairdressing salon, in which a temperature in the range of from about 10-29° C. prevails.

The hair coloring application mixture can also be left on the fibers, in particular the hair, at least about 30° C., preferably at from about 30-60° C., more preferably at from about 32-50° C., when the hair, for example, is heated with a heat hood or with a heat radiator.

The oxidizing agent preparation (K2) used in color kits as contemplated herein and preferred as contemplated herein and in dyeing methods as contemplated herein and preferred as contemplated herein contains, in each case its by weight, preferably from about 40-96% by weight, more preferably from about 70-93% by weight, most preferably from about 80%-90% by weight of water.

The oxidizing agent preparation (K2) used in preferred color kits as contemplated herein as well as in preferred dyeing methods as contemplated herein further contains, in each case by its weight, preferably from about 0.5 to about 23% by weight, more preferably from about 2.5 to about 21% by weight, further preferably from about 4 to about 20% by weight, yet more preferably from about 5 to about 18% by weight and most preferably from about 6 to about 12% by weight of hydrogen peroxide.

To stabilize the hydrogen peroxide, the oxidizing agent preparation (K2) preferably has a pH value in the range from about 2.0 to about 6.5, particularly preferably from about 2.5-5.5, most preferably from about 2.8 to about 5.0, measured in each case at about 20° C.

Cationic surfactant in the oxidizing agent preparation (K2)

The oxidizing agent preparation (K2) usually has a viscosity in the range from about 10-6000 mPas, preferably from about 200-5000 mPas, particularly preferably from about 1000-4500 mPas, in each case measured at about 20° C. For the application to the hair, however, the application mixture should have a significantly higher viscosity, so that it remains on the hair throughout the contact time (in the range of from about 5-60 minutes, preferably from about 30-45 minutes) and does not drip down. Here, a distinction is made as to whether the application mixture is prepared by shaking both compositions (K1) and (K2) in an application bottle from which the application mixture is applied to the hair immediately after mixing with the aid of an application nozzle as a bottle attachment (bottle application), or whether the application mixture is prepared by stirring both compositions (K1) and (K2) in a dish from which the application mixture is applied to the hair immediately after mixing with a brush (brush application). The bottle application is particularly suitable for coloring agents that are sold retail with a recommendation for use by the consumer himself. The brush application is particularly suitable for coloring agents that are prepared in the hairdressing salon by the hairdresser and applied to the hair of the consumer.

It has surprisingly been found that an application mixture having a viscosity suitable in particular for brush application is obtained by mixing a component (K1) as contemplated herein or preferred as contemplated herein with an oxidizing agent preparation (K2) containing at least one cationic surfactant, in particular when (K1) contains an anionic surfactant. During mixing, the interaction between anionic constituents in (K1) and the at least one cationic surfactant leads to the desired increase in viscosity. The thus resulting pasty consistency of the application mixture leads to optimal application properties, in particular for brush application. The application mixtures thus obtained, in particular the mixtures whose weight-related mixing ratio (K1):(K2) is in the range from about 1:0.8 to about 1:2.5, more preferably in the range from about 1:1 to about 1:2, preferably have a viscosity in the range from about 20,000 to about 100,000 mPas, preferably from about 30,000 to about 80,000 mPas, particularly preferably from about 45,000 to about 70,000 mPas, in each case measured at about 20° C. (Brookfield viscometer, rotational frequency of about 4 min⁻¹, spindle no. 5).

In a further preferred embodiment, the oxidizing agent preparation (K2) contains at least one cationic surfactant, preferably in a total amount of from about 0.05 to about 3% by weight, particularly preferably from about 0.1 to about 1.5% by weight, most preferably from about 0.3 to about 0.9% by weight, in each case based on the weight of the oxidizing agent preparation (K2).

Cationic surfactants are understood to mean surfactants, that is, surface-active compounds, each having one or more positive charges. Cationic surfactants contain only positive charges. Usually, these surfactants are composed of a hydrophobic part and a hydrophilic head group, wherein the hydrophobic part usually includes a hydrocarbon skeleton (for example, including one or two linear or branched alkyl chains), and the positive charge(s) are localized in the hydrophilic head group. Cationic surfactants adsorb at interfaces and aggregate in aqueous solution above the critical micelle concentration to positively charged micelles.

As contemplated herein, preference is given to cationic surfactants of the type of quaternary ammonium compounds, esterquats and amidoamines. Preferred quaternary ammonium compounds are ammonium halides such as alkyltrimethylammonium chlorides, dialkyldimethylammonium chlorides, trialkylmethylammonium chlorides, and the imidazolium compounds known under the INCI names Quaternium-27 and Quaternium-83. Further preferred quaternary ammonium compounds are tetraalkylammonium salts, such as in particular the quaternium-52 known under the INCI name, a poly (oxy-1,2-ethanediyl), ((octadecylnitrilio) tri-2,1-ethanediyl) tris (hydroxy) phosphate (1:1) salt having the general structural formula (III) wherein x+y+z=10:

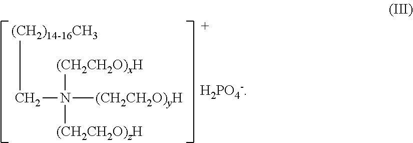

The long alkyl chains of the aforementioned surfactants preferably have 10 to 22, more preferably 12 to 18 carbon atoms. Particularly preferred are behenyltrimethylammonium chloride, stearyltrimethylammonium chloride and cetyltrimethylammonium chloride, wherein stearyltrimethylammonium chloride is highly preferred. Further suitable cationic surfactants are quaternized protein hydrolyzates. Alkylamidoamines are usually prepared by amidation of natural or synthetic fatty acids and fatty acid cuts with dialkylaminoamines. A compound suitable as contemplated herein suitable from this group of substances constitutes Tegoamid® S 18 (stearamidopropyl dimethylamine). Esterquats are substances which contain both at least one ester function and at least one quaternary ammonium group as a structural element. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are sold, for example, under the trademarks Stepantex, Dehyquart and Armocare.

C10-C22 alkyltrimethylammonium chlorides have proven to be particularly suitable with regard to optimum application properties and optimum dyeing results. Particularly preferred oxidizing agent preparations (K2) used as contemplated herein contain at least one cationic surfactant in a total amount of from about 0.05 to about 3% by weight, particularly preferably from about 0.1 to about 1.5% by weight, most preferably from about 0.3 to about 0.9% by weight, in each case based on the weight of the oxidizing agent preparation (K2), wherein preferably at least one surfactant selected from C10-C22-alkyltrimethylammonium chlorides, in particular selected from behenyltrimethylammonium chloride, stearyltrimethylammonium chloride, cetyltrimethylammonium chloride, and mixtures of these surfactants is present. Extremely preferred oxidizing agent preparations (K2) used as contemplated herein contain stearyltrimethylammonium chloride in a total amount of from about 0.05 to about 3% by weight, more preferably from about 0.1-1.5% by weight, even more preferably from about 0.3-0.9%, in each case based on the weight of the oxidizing agent preparation K2.

A further preferred packaging unit (kit-of-parts) as contemplated herein is exemplified in that the oxidizing agent preparation (K2) contains at least one cationic surfactant, preferably in a total amount of from about 0.05 to about 3% by weight, particularly preferably from about 0.1 to about 1.5% by weight, most preferably from about 0.3 to about 0.9% by weight, in each case based on the weight of the oxidizing agent preparation (K2), but no polymer having a degree of polymerization of at least about 200 and no polymer having a molecular weight of about 10,000 daltons or higher.

A further preferred packaging unit (kit-of-parts) as contemplated herein is exemplified in that the oxidizing agent preparation (K2) contains at least one cationic surfactant, which is preferably selected from stearyl trimethyl ammonium chloride, in a total amount of from about 0.05-3% by weight, particularly preferably from about 0.1-1.5% by weight, most preferably from about 0.3-0.9% by weight, in each case based on the weight of the oxidizing agent preparation (K2), but no polymer having a degree of polymerization of at least about 200 and no polymer having a molecular weight of about 10,000 daltons or higher.

A method for oxidative hair coloring preferred as contemplated herein is exemplified in that the oxidizing agent preparation (K2) contains at least one cationic surfactant, preferably in a total amount of from about 0.05-3% by weight, particularly preferably from about 0.1-1.5% by weight, most preferably from about 0.3 to about 0.9% by weight, in each case based on the weight of the oxidizing agent preparation (K2), but no polymer having a degree of polymerization of at least about 200 and no polymer having a molecular weight of about 10,000 daltons or higher.

A further method for oxidative hair coloring preferred as contemplated herein is exemplified in that the oxidizing agent preparation (K2) contains at least one cationic surfactant, which is preferably selected from stearyl trimethyl ammonium chloride, in a total amount of from about 0.05-3% by weight, particularly preferably from about 0.1-1.5% by weight, most preferably from about 0.3-0.9% by weight, in each case based on the weight of the oxidizing agent preparation (K2), but no polymer having a degree of polymerization of at least about 200 and no polymer having a molecular weight of about 10,000 daltons or higher.

Surprisingly, it has been found that an application mixture having a viscosity suitable in particular for bottle application is obtained by mixing a component (K1) as contemplated herein or preferred as contemplated herein with an oxidizing agent preparation (K2) which contains at least one copolymer selected from crosslinked acrylic acid/acrylic acid C1-C6 alkyl ester copolymers and crosslinked methacrylic acid/acrylic acid C1-C6 alkyl ester copolymers, preferably, in a total amount of from about 0.1-7% by weight, particularly preferably from about 0.5-6% by weight, extremely preferably from about 1-4.5% by weight, in each case based on the weight of the oxidizing agent preparation (K2). The mixing of the agent as contemplated herein or preferred as contemplated herein with such an oxidizing agent preparation (K2) leads to the desired increase in viscosity. The thus resulting medium-viscosity consistency of the application mixture leads to optimal application properties, in particular for bottle application. The application mixtures thus obtained, in particular the mixtures with weight-related mixing ratios (K1):(K2), is in the range from about 1:0.8 to about 1:2.5, more preferably in the range from about 1:1 to about 1:2, preferably have a viscosity in the range from about 10,000-50,000 mPas, preferably about 15,000-30,000 mPas, particularly preferably from about 18,000-25,000 mPas, in each case measured at about 20° C. (Brookfield viscometer, rotational frequency of about 4 min⁻¹, spindle no. 5).

A further packaging unit (kit-of-parts) preferred as contemplated herein is therefore exemplified in that the oxidizing agent preparation (K2) contains at least one copolymer selected from crosslinked acrylic acid/acrylic acid C1-C6 alkyl ester copolymers and crosslinked methacrylic acid/acrylic acid C1-C6 alkyl ester copolymers, preferably in a total amount of from about 0.1-7% by weight, particularly preferably from about 0.5-6% by weight, most preferably from about 1-4.5% by weight, in each case based on the weight of the oxidizing agent preparation (K2), and preferably contains no cationic surfactant.

A further method for oxidative hair coloring preferred as contemplated herein is therefore exemplified in that the oxidizing agent preparation (K2) contains at least one copolymer selected from crosslinked acrylic acid/acrylic acid C1-C6 alkyl ester copolymers and crosslinked methacrylic acid/acrylic acid C1-C6 alkyl ester copolymers, preferably in a total amount of from about 0.1-7% by weight, particularly preferably from about 0.5-6% by weight, most preferably from about 1-4.5% by weight, in each case based on the weight of the oxidizing agent preparation (K2), and preferably contains no cationic surfactant.

Preferred crosslinked copolymers of this type are selected from, in each case crosslinked, methacrylic acid/methyl acrylate, methacrylic acid/ethyl acrylate, methacrylic acid/propyl acrylate, methacrylic acid/butyl acrylate, methacrylic acid/pentyl acrylate, methacrylic acid/hexyl acrylate, acrylic acid/methyl acrylate, acrylic acid/ethyl acrylate, acrylic acid/propyl acrylate, acrylic acid/butyl acrylate, acrylic acid/pentyl acrylate and acrylic acid/hexyl acrylate copolymers and mixtures thereof.

A further preferred packaging unit (kit-of-parts) as contemplated herein is characterized in that the oxidizing agent preparation (K2) contains at least one crosslinked copolymer selected from, in each case crosslinked, methacrylic acid/methyl acrylate, methacrylic acid/ethyl acrylate, methacrylic acid/propyl acrylate, methacrylic acid/butyl acrylate, methacrylic acid/pentyl acrylate, methacrylic acid/hexyl acrylate, acrylic acid/methyl acrylate, acrylic acid/ethyl acrylate, acrylic acid/propyl acrylate, acrylic acid/butyl acrylate, acrylic acid/pentyl acrylate and acrylic acid/hexyl acrylate copolymers and mixtures thereof, in a total amount from about 0.1-7% by weight, particularly preferably from about 0.5-6% by weight, most preferably from about 1-4.5% by weight, in each case based on the weight of the oxidizing agent preparation (K2), and no cationic surfactant.

A further method for oxidative hair coloring preferred as contemplated herein is exemplified in that the oxidizing agent preparation (K2) contains at least one crosslinked copolymer selected from, in each case crosslinked, methacrylic acid/methyl acrylate, methacrylic acid/ethyl acrylate, methacrylic acid/propyl acrylate, methacrylic acid/butyl acrylate, methacrylic acid/pentyl acrylate, methacrylic acid/hexyl acrylate, acrylic acid/methyl acrylate, acrylic acid/ethyl acrylate, acrylic acid/propyl acrylate, acrylic acid/butyl acrylate, acrylic acid/pentyl acrylate and acrylic acid/hexyl acrylate copolymers and mixtures thereof, in a total amount from about 0.1-7% by weight, particularly preferably from about 0.5-6% by weight, most preferably from about 1-4.5% by weight, in each case based on the weight of the oxidizing agent preparation (K2), and no cationic surfactant.

The oxidizing agent preparations (K2) used as contemplated herein and preferably used as contemplated herein can additionally contain stabilizers, in particular complexing agents, and pH buffer substances.

In a further preferred embodiment of the present disclosure, the oxidizing agent preparation (K2) used as contemplated herein contains at least one oil in a total amount of from about 0.2-50% by weight, preferably from about 2-40% by weight, particularly preferably from about 8-30% by weight, most preferably from about 15-25% by weight, in each case based on the weight of the oxidizing agent preparation (K2).

In a particularly preferred embodiment of the present disclosure, the oxidizing agent preparation (K2) used as contemplated herein contains no cationic surfactant and at least one oil in a total amount of from about 0.2-50% by weight, more preferably from about 2-40% by weight, most preferably from about 8%-30% by weight, more preferably from about 15-25% by weight, in each case based on the weight of the oxidizing agent preparation (K2).

The at least one oil contained in the oxidizing agent preparation (K2) in a total amount of from about 0.2-50% by weight, based on the weight of the preparation (K2), is preferably selected from natural and synthetic hydrocarbons, more preferably from paraffin oils, C₁₈-C₃₀ isoparaffins, in particular isoeicosane, polyisobutenes and polydecenes, C₈-C₁₆ isoparaffins, and 1,3-di-(2-ethylhexyl) cyclohexane; the benzoic acid esters of linear or branched C₈₋₂₂ alkanols; triglycerides of linear or branched, saturated or unsaturated, optionally hydroxylated C₈₋₃₀ fatty acids, in particular natural oils; the dicarboxylic acid esters of linear or branched C₂-C₁₀ alkanols; the esters of linear or branched saturated or unsaturated fatty alcohols having 2-30 carbon atoms with linear or branched saturated or unsaturated fatty acids having 2-30 carbon atoms which can be hydroxylated; the addition products of 1 to 5 propylene oxide units on monovalent or polyvalent C₈₋₂₂ alkanols; the C₈-C₂₂ fatty alcohol esters of monovalent or polyvalent C₂-C₇ hydroxycarboxylic acids; the symmetric, unsymmetrical or cyclic esters of carbonic acid with C₃₋₂₂ alkanols, C₃₋₂₂ alkane diols or C₃₋₂₂ alkane triols; the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols; silicone oils and mixtures of the aforementioned substances. In this context, particularly preferred oils as contemplated herein are selected from paraffin oils and the esters of linear or branched saturated or unsaturated fatty alcohols having 2-30 carbon atoms with linear or branched saturated or unsaturated fatty acids having 2-30 carbon atoms which can be hydroxylated, and mixtures thereof; most preferably selected from paraffin oil, isopropyl palmitate and isopropyl myristate and mixtures thereof.

In a further preferred embodiment of the present disclosure, the oxidizing agent preparation (K2) used as contemplated herein contains at least one surfactant selected from anionic surfactants and nonionic surfactants and mixtures thereof in a total amount of from about 0.05-2% by weight, preferably from about 0.3-1.5% by weight, and at least one linear, saturated 1-alkanol having 14 to 22 carbon atoms, selected from 1-tetradecanol (myristyl alcohol), 1-hexadecanol (cetyl alcohol), 1-octadecanol (stearyl alcohol) and 1-eicosanol (arachyl alcohol) and mixtures thereof, in a total amount of from about 1-5% by weight, preferably from about 1.5-4% by weight, wherein all amount specifications are based on the weight of the oxidizing agent preparation (K2), and wherein the preparation (K2) contains no cationic surfactants, no oils, no polymer having a degree of polymerization of at least about 200 and no polymer having a molecular weight of about 10,000 daltons or higher.

A further preferred kit as contemplated herein (kit-of-parts) and a further hair-coloring method are each exemplified in that the oxidizing agent preparation (K2) contains at least one surfactant selected from anionic surfactants and nonionic surfactants and mixtures thereof, in a total amount of from about 0.05-2% by weight, preferably from about 0.3-1.5% by weight, and at least one linear, saturated 1-alkanol having 14 to 22 carbon atoms, selected from 1-tetradecanol (myristyl alcohol), 1-hexadecanol (cetyl alcohol), 1-octadecanol (stearyl alcohol) and 1-eicosanol (arachyl alcohol) and mixtures thereof, in a total amount of from about 1-5% by weight, preferably from about 1.5-4% by weight, in each case based on the weight of the oxidizing agent preparation (K2).

A further preferred kit as contemplated herein (kit-of-parts) and a further hair-coloring method are each exemplified in that the oxidizing agent preparation (K2) contains at least one surfactant selected from anionic surfactants and nonionic surfactants and mixtures thereof, in a total amount of from about 0.05-2% by weight, preferably from about 0.3-1.5% by weight, and at least one linear, saturated 1-alkanol having 14 to 22 carbon atoms, selected from 1-tetradecanol (myristyl alcohol), 1-hexadecanol (cetyl alcohol), 1-octadecanol (stearyl alcohol) and 1-eicosanol (arachyl alcohol) and mixtures thereof, in a total amount of from about 1-5% by weight, preferably from about 1.5-4% by weight, in each case by weight of the oxidizing agent preparation (K2) but no polymer having a degree of polymerization of at least about 200 and no polymer having a molecular weight of about 10,000 daltons or higher.

It has been found that the thickening through the aid of the interaction between the copolymer in the agent as contemplated herein and the aforementioned surfactant/1-alkanol mixture in the oxidizing agent preparation (K2) is sufficient and the presence of a polymer having a degree of polymerization of at least about 200 or a polymer having a molecular weight of about 10,000 daltons or higher cannot further increase or even impair in their application properties.

A further preferred kit as contemplated herein (kit-of-parts) and a further hair-coloring method as contemplated herein are each exemplified in that the oxidizing agent preparation (K2) contains at least one surfactant selected from anionic surfactants and nonionic surfactants and mixtures thereof, in a total amount of from about 0.05-2% by weight, preferably from about 0.3-1.5% by weight, at least one linear, saturated 1-alkanol having 14 to 22 carbon atoms, selected from 1-tetradecanol (myristyl alcohol), 1-hexadecanol (cetyl alcohol), 1-octadecanol (stearyl alcohol) and 1-eicosanol (arachyl alcohol) and mixtures thereof, in a total amount of from about 1-5% by weight, preferably from about 1.5-4% by weight, and at least one oil in a total amount of from about 0.2-50% by weight, preferably from about 2-40% by weight, particularly preferably from about 8-30% by weight, most preferably from about 15-25% by weight, in each case based on the weight of the oxidizing agent preparation (K2).

A further preferred kit as contemplated herein (kit-of-parts) and a further hair-coloring method as contemplated herein are each exemplified in that the oxidizing agent preparation (K2) contains at least one surfactant selected from anionic surfactants and nonionic surfactants and mixtures thereof, in a total amount of from about 0.05-2% by weight, preferably from about 0.3-1.5% by weight, at least one linear, saturated 1-alkanol having 14 to 22 carbon atoms, selected from 1-tetradecanol (myristyl alcohol), 1-hexadecanol (cetyl alcohol), 1-octadecanol (stearyl alcohol) and 1-eicosanol (arachyl alcohol) and mixtures thereof, in a total amount of from about 1-5% by weight, preferably from about 1.5-4% by weight, and at least one oil in a total amount of from about 0.2-50% by weight, preferably from about 2-40% by weight, particularly preferably from about 8-30% by weight, most preferably from about 15-25% by weight, in each case based on the weight of the oxidizing agent preparation (K2), but no polymer having a degree of polymerization of at least 200 and no polymer having a molecular weight of about 10,000 daltons or higher.

Suitable anionic surfactants for the oxidizing agent preparations (K2) used as contemplated herein are all anionic surfactants which have been discussed above for the agents as contemplated herein as a whole.

Suitable nonionic surfactants for the oxidizing agent preparations (K2) used as contemplated herein are all nonionic surface-active substances suitable for use on the human body, which have been discussed above for the agents as contemplated herein as a whole.

The following examples are intended to illustrate the subject matter of the present disclosure without, however, limiting it.

### 1.1. Preparation of the Coloring Agents

The following color creams have been prepared (all specifications are in percent by weight unless otherwise stated):

| | Cream No. 1 | |
|---|---|---|
| | Component (K1) of the | Cream No. 2 |
| | fourth subject of the present | Component (K1) of the |
| | disclosure or component | second subject of the |
| | (K1) comparison | present disclosure |
| 1-hydroxyethyl 4,5- | 1.50 | 1.50 |
| diaminopyrazole sulfate | | |
| 4-amino-2-hydroxytoluene | 0.30 | 0.30 |
| 4-amino-m-cresol | 0.18 | 0.18 |
| m-aminophenol | 0.60 | 0.60 |
| Tetrasodium iminodisuccinate | — | 1.94 (salt) = 1.43 (acid) |
| Ammonium hydroxide | 3.20 | 3.20 |
| Monoethanolamine | 0.60 | 0.60 |
| Potassium hydroxide | 0.06 | 0.06 |
| Octyldodecanol | 1.60 | 1.60 |
| Cetearyl alcohol | 9.60 | 9.60 |
| Glyceryl stearate (self- | 3.00 | 3.00 |
| emulsifying) | | |
| Ceteareth-20 | 2.40 | 2.40 |
| Sodium laureth sulfate | 0.90 | 0.90 |
| Sodium cetearyl sulfate | 0.50 | 0.50 |
| Oleic acid | 0.30 | 0.30 |
| Sodium sulfite | 0.40 | 0.40 |
| Perfume | 0.30 | 0.30 |
| Propylene glycol | 0.02 | 0.02 |
| Glycerol | 0.20 | 0.20 |
| Tetrasodium EDTA | 0.20 | 0.20 |
| Carbomer | 0.12 | 0.12 |
| Ascorbic acid | 0.05 | 0.05 |
| Sodium sulphate | 0.02 | 0.02 |
| Sodium benzoate | 0.01 | 0.01 |
| Water | 73.94 | 72.00 |

### Oxidizing Agent Component (K2)

| | |
|---|---|
| Hydrogen peroxide | 6.0 |
| Cetearyl alcohol | 1.6 |
| PEG-40 Castor Oil | 0.3 |
| Disodiumpyrophosphate | 0.3 |
| Sodium cetearyl sulfate | 0.2 |
| Disodium EDTA | 0.1 |
| Sodium benzoate | 0.04 |
| Phosphoric acid | 0.03 |
| Water | 91.43 |

### Component (K3) of the Fourth Subject of the Present Disclosure

| Tetrasodium iminodisuccinate | 8.00 (salt) = 5.91 (acid) |
|---|---|
| Polyvinylpyrrolidone with K value | 10.00 active substance |
| 27-33 | |
| Phenoxyethanol | 0.60 |
| Ethylhexylglycerin | 0.09 |
| Keratin hydrolyzate | 0.01 |
| Water | 81.30 |

The comparative coloring agent (V1) was prepared by a mixture of cream no. 1 and the oxidizing agent component (K2) each in equal parts by weight (1:1).

A coloring agent (E1) according to the first aspect of the present disclosure was prepared by a mixture of cream no. 2 (as component (K1) of the second subject of the present disclosure) and the oxidizing agent component (K2) in equal parts by weight (1:1).

### 1.2 Dyeing on the Keratin Fibers

Kerling 10-0 untreated hair strands (about 1 gram) were treated as follows. The aforementioned coloring agent (V1) was applied directly to the hair strands mentioned after its preparation (liquor ratio 4 grams of ready-to-use coloring agent (V1) per gram of hair) and left on the hair for a contact time of 30 minutes at room temperature (22° C.). Subsequently, the coloring agent was rinsed from the strands with water. The strands were first dried with a towel and then in a cold air stream. All strands were intensely colored red.

The colorimetric L*a*b* values for these strands formed the target values, that is, the desired shade.

### 1.3 Measurement of Color Shift Due to Copper Ions

For the evaluation of the influence of copper ions on the color shift of the dyeing, further hair strands of the brand Kerling 10-0 were immersed for 60 seconds in a copper (II) salt solution, then rinsed with water, dried first with a towel and then in a cold air stream. A part of the copper ion-containing strands was dyed with the comparative coloring agent (V1), and the other part of the copper ion-containing strands was dyed with the coloring agent (E1) of the present disclosure, each by the method shown in 1.2.

Color measurements were made according to the L*a*b* color system to determine the color shift.

For each of the 3 staining experiments (1. Copper-free strand with (V1), 2. Copper-containing strand with (V1), 3. Copper-containing strand with (E1)), three strands each of hair were used and the arithmetical mean calculated for all hair strands. From the measured L*a*b* values, the color difference ΔE between the color of the dyed, copper-free hair (value combination L₀*, a₀*, b₀*) and the color of the dyed, copper-containing hair (value combination Lᵢ*, aᵢ*, bᵢ*) was calculated according to the following formula: ΔE=((Lᵢ−L₀)²+(aᵢ−a₀)²+(bᵢ−b₀)²)^{1/2}

The larger the value for ΔE, the more pronounced is the color difference. Color differences with ΔE<1 are imperceptible to the human eye. Color differences with ΔE<2 are only visible to the trained eye. Color differences with ΔE>2 are also visible to the untrained eye.

### 1.4 Results of Color Measurements for Color Shift:

| Dyeing experiment no. | ΔE |
|---|---|
| 1. Copper-free strand with (V1) | Reference |
| 2. Copper-containing strand with (V1) | 9.0 |
| 3. Copper-containing strand with (E1) | 2.3 |

A significantly reduced color shift compared to the color of the copper-free strands is achieved with the coloring agent (E1) as contemplated herein, which contains tetrasodium iminodisuccinate, than with the non-inventive coloring agent (V1) without tetrasodium iminodisuccinate. The coloring achieved with the tetrasodium iminodisuccinate-containing coloring agent differs from the reference color of the copper-free strands to an extent barely perceptible to the naked eye.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An agent for the oxidative dyeing of keratinic fibers, comprising in a cosmetic carrier
(A) at least one oxidation dye precursor selected from compounds of structure (I) and salts thereof 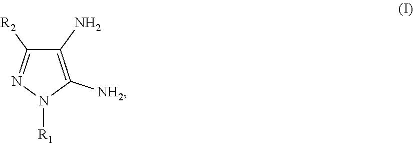
wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen;
(B) iminodisuccinic acid or a salt thereof; and
(C) at least one oxidizing agent other than atmospheric oxygen.

2. The agent according to claim 1, wherein R₂ is hydrogen.

3. The agent according to claim 1, comprising 4,5-diamino-1-(2-hydroxyethyl)-1H-pyrazole or a salt thereof.

4. The agent according to claim 1, wherein R₁ is n-hexyl or n-heptyl group and R₂ is hydrogen.

5. The agent according to claim 1, comprising a sodium salt of iminodisuccinic acid.

6. The agent according to claim 1, comprising, based on its total weight, iminodisuccinic acid or a salt thereof in a total amount of about 0.01 to to about 3% by weight, wherein the amount specifications are based on the weight converted to free iminodisuccinic acid in relation to the weight of the agent.

7. The agent according to claim 1, wherein the at least one oxidizing agent other than atmospheric oxygen is selected from hydrogen peroxide, sodium percarbonate, percarbonates and persalts.

8. A multi-component packaging unit (kit-of-parts) for the oxidative dyeing of keratinic fibers, comprising at least two separately prepared components (K1) and (K2), wherein
the first component (K1), in a cosmetic carrier, comprises
(A) at least one oxidation dye precursor selected from compounds of structure (I) and salts thereof 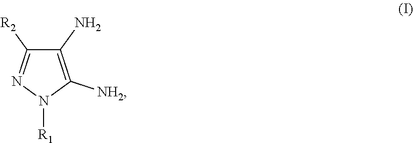
wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, wherein the component (A) is selected according to claim 1, and
(C) optionally at least one oxidizing agent selected from persalts, and
the second component (K2) comprises hydrogen peroxide dissolved in water,
wherein at least one of the components (K1) or (K2)
comprises iminodisuccinic acid or a salt thereof.

9. A multi-component packaging unit (kit-of-parts) for the oxidative dyeing of keratinic fibers, comprising at least two separately prepared components (K1) and (K2), wherein
the first component (K1), in a cosmetic carrier, comprises
(A) at least one oxidation dye precursor selected from compounds of the structure (I) and salts thereof 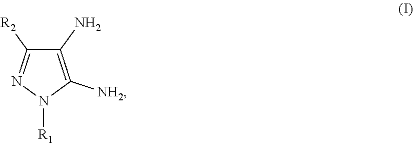
wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, wherein the component (A) is selected according to claim 1, and
sodium percarbonate and optionally at least one further oxidizing agent selected from persalts, and wherein
the second component (K2) comprises water,
wherein at least one of the components (K1) and (K2) comprises (B) iminodisuccinic acid or a salt thereof.

10. A multi-component packaging unit (kit-of-parts) for the oxidative dyeing of keratinic fibers, comprising at least three separately prepared components (K1), (K2) and (K3), wherein
the first component (K1), in a cosmetic carrier,
(A) comprises at least one oxidation dye precursor selected from compounds of structure (I) and salts thereof 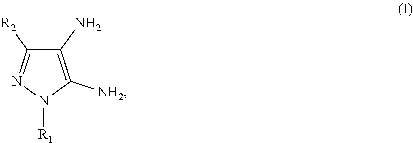
in which R₁ and R₂, independently of one another, stand for hydrogen or a linear or branched C1-C10 alkyl group which can be substituted by one to ten hydroxyl groups, wherein R₁ and R₂ do not simultaneously stand for hydrogen, wherein the component (A) is selected according to claim 1, and
at least one oxidizing agent selected from persalts, and wherein
the second component (K2)
comprises hydrogen peroxide dissolved in water, and wherein the third component (K3) comprises iminodisuccinic acid or a salt thereof.

11. A multi-component packaging unit (kit-of-parts) for the oxidative dyeing of keratinic fibers, comprising at least three separately prepared components (K1), (K2) and (K3), wherein
the first component (K1), in a cosmetic carrier, comprises
at least one oxidation dye precursor selected from compounds of structure (I) and salts thereof 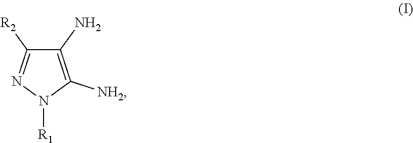
wherein R₁ and R₂, independently of one another, stand for hydrogen or a linear or branched C1-C10 alkyl group which can be substituted by one to ten hydroxyl groups, wherein R₁ and R₂ do not simultaneously stand for hydrogen, wherein the component (A) is selected according to claim 1;
sodium percarbonate and optionally at least one further oxidizing agent selected from persalts;
the second component (K2) comprises water; and
the third component (K3) comprises iminodisuccinic acid or a salt thereof.

12. A method for the oxidative hair dyeing of keratinic fibers, in which an agent according to claim 1 is applied to the fibers, left there on the fibers for a period of from about 1 to about 60 minutes, at room temperature or at least about 30° C., and then the fibers, are rinsed with water.

13. A method for the oxidative hair dyeing of keratinic fibers, in which the components of the kits according to claim 8 are applied on the fibers simultaneously or directly after one another, and without rinsing left there on the fibers for a period of from about 1 to about 60 minutes, at room temperature or at least about 30° C., are then rinsed with water or a cleaning composition.

14. A method for the oxidative hair dyeing of keratinic fibers, in which the components of the kits according to claim 9 are applied on the fibers simultaneously or directly after one another, and without rinsing left there on the fibers for a period of from about 1 to about 60 minutes, at room temperature or at least about 30° C., are then rinsed with water or a cleaning composition.

15. A method for the oxidative hair dyeing of keratinic fibers, in which the components of the kits according to claim 10 are applied on the fibers simultaneously or directly after one another, and without rinsing left there on the fibers for a period of from about 1 to about 60 minutes, at room temperature or at least about 30° C., are then rinsed with water or a cleaning composition.

16. A method for the oxidative hair dyeing of keratinic fibers, in which the components of the kits according to claim 8 are applied on the fibers simultaneously or directly after one another, and without rinsing left there on the fibers for a period of from about 1 to about 60 minutes, at room temperature or at least about 30° C., are then rinsed with water or a cleaning composition.
